(19) United States
(12) Patent Application Publication
(10) Pub. No.: US 2022/0000853 A1
(43) Pub. Date: Jan. 6, 2022

(54) FORMULATIONS, METHODS, KIT, AND DOSAGE FORMS FOR IMPROVED STABILITY OF AN ACTIVE PHARMACEUTICAL INGREDIENT

(71) Applicant: Asana BioSciences, LLC, Lawrenceville, NJ (US)

(72) Inventors: Jay Audett, Bridegwater, NJ (US); Tarun Goswani, Bridegwater, NJ (US); Sameer Suchdeva, Bridgewater, NJ (US); Scott K. Thompson, Bridgewater, NJ (US); Padam Bansal, Bridgewater, NJ (US); Niranjan Rao, Bridgewater, NJ (US); Pablo Jimenez, Bridgewater, NJ (US)

(21) Appl. No.: 17/296,617

(22) PCT Filed: Nov. 27, 2019
(86) PCT No.: PCT/US2019/063671

§ 371 Date: May 25, 2021

Related U.S. Application Data
(60) Provisional application No. 62/773,277, filed on Nov. 30, 2018.
(60) Provisional application No. 62/840,726, filed on Apr. 30, 2019.

(51) Int. Cl. A61K 31/452; A61K 9/00; A61K 47/34; A61P 17/04; A61K 31/235

(52) U.S. Cl. CPC A61K 31/452; A61K 9/0014; A61K 31/235; A61P 17/04; A61K 47/34

(57) ABSTRACT

Embodiments of the disclosure relate generally to formulations, methods, kits, and dosage forms for improved topical pharmaceutical formulation comprising an active ingredient, wherein the active ingredient comprises a compound selected from the group consisting of the Formula (I), Formula (II), and Formula (III): The formulations can further comprise a hydrophilic non-ionic surfactant comprising a poloxamer. These formulations are useful in treating inflammation, pruritus and/or pain, or for treating conditions for which the signs and symptoms include inflammation, pruritis and/or pain, by topical administration to a subject.

### TECHNICAL FIELD

Embodiments of the disclosure relate generally to formulations, methods, kits, and dosage forms of an improved topical pharmaceutical formulation that can be used in the treatment of adverse conditions, including for the treatment of itch and/or pain.

### BACKGROUND

Local anesthetics such as lidocaine are useful in pain relief in numerous applications, but suffer from the drawback of undesired blockade of motor function. This is because such local anesthetics are non-selective sodium channel blockers that fail to discriminate between sodium channel activity required for normal ongoing sensation and similar activity involved in nociceptor signaling. The cationic sodium channel blocker, QX-314, selectively blocks sodium channel activity in nociceptor neurons when administered in the presence of capsaicin, an agonist for the transient receptor potential cation channel, subfamily V, member 1 (TRPV1). TRPV1 is preferentially expressed peripherally in small-diameter primary afferent nociceptors and is up-regulated in chronic pain states. However, TRPV1 is not present in the large diameter afferent neurons that convey tactile sensations nor is TRPV1 present in motor neuron efferent fibers.

QX-314 is the N-ethylated analog of lidocaine and bears a permanent positive charge. It is unable to cross the neuronal cell membrane when applied externally and has no effect on neuronal sodium-channel activity unless afforded access to the cell cytoplasm through open TRPV1 channels in which case it causes prolonged block of sodium-channel activity. Voltage-clamp single cell electrophysiology experiments illustrated that QX-314 permeates through capsaicin-activated TRPV1 channels and blocks sodium channel activity. In vivo, perisciatic administration of a QX-314/capsaicin combination produced pronounced and long-lasting nociceptor-selective nerve blockade.

There remains a need in the art for topical pharmaceutical formulations which are useful in the management of long-term or chronic pain and compounds for pain management which minimize impairment of motor function. Furthermore, there remains a need in the art for topical pharmaceutical formulations which are useful for the treatment of dermatological conditions including itch, and/or pain.

### SUMMARY

The present disclosure relates to topical formulations, methods, kits, and dosage forms for treating inflammation, pruritus and/or pain, or for treating conditions for which the signs and symptoms include inflammation, pruritis and/or pain. In an embodiment, the present disclosure provides a pharmaceutical formulation comprising an active ingredient in substantially gel-like form and a hydrophilic non-ionic surfactant comprising a poloxamer, wherein the active ingredient is a compound selected from the group consisting of Formula (I), Formula (II), and Formula (III), or a pharmaceutically acceptable salt or prodrug thereof,

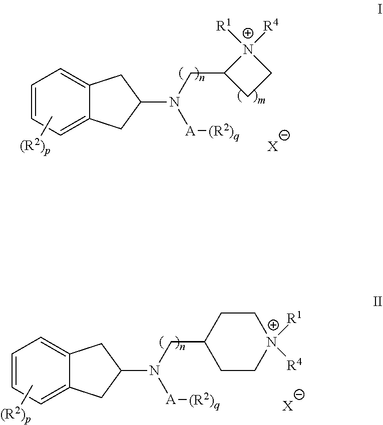

wherein: A is phenyl or heteroaryl; R¹ and R⁴ are, independently, C₁ to C₆ alkyl or CH₂CH₂OH; or R¹ and R⁴ are joined to form a 4- or 6-membered carbocyclic or heterocyclic ring; R² is independently selected from the group consisting of hydrogen, halogen, NO₂, OH, and C₁ to C₆ alkoxy; R³ is independently selected from the group consisting of hydrogen, halogen, CN, NO₂, NH₂, optionally substituted C₁ to C₆ alkyl, C₂ to C₆ alkenyl, C₂ to C₆ alkynyl, OH, CF₃, OCF₃, SCF₃, optionally substituted C₁ to C₆ alkoxy, C₂ to C₆ alkynyloxy, heterocyclyloxy, heteroaryloxy, optionally substituted C₁ to C₆ alkylthio, heteroarylthio, C(O)O(C₁ to C₆ alkyl), C(O)(C₁ to C₆ alkyl), C(O)(aryl), C(O)(heterocycle), C(O)NH₂, C(O)NH(C₁ to C₆ alkyl), C(O)NH(aryl), C(O)NH(heterocycle), C(O)NH(heteroaryl), C(O)N(C₁ to C₆ alkyl)(C₁ to C₆ alkyl), C(O)N(aryl)(C₁ to C₆ alkyl), C(S)NH₂, optionally substituted aryl, heteroaryl, heterocycle, NHC(O)(C₁ to C₆ alkyl), NHC(O)(aryl), NHC(O)(heteroaryl), NHC(O)O(C₁ to C₆ alkyl), N(C₁ to C₆ alkyl)C(O)(C₁ to C₆ alkyl), N(C₁ to C₆ alkyl)C(O)O(C₁ to C₆ alkyl), NHC(O)NH₂, NHC(O)NH(C₁ to C₆ alkyl), NHC(O)NH(heteroaryl), NHSO₂(C₁ to C₆ alkyl), SO₂(C₁ to C₆ alkyl), SO₂NH₂, SO₂NH(C₁ to C₆ alkyl), SO₂NH(C₂ to C₆ alkynyl), SO₂N(C₁ to C₆ alkyl)(C₁ to C₆ alkyl), SO₂NH(heteroaryl), NH(C₁ to C₆ alkyl), N(C₁ to C₆ alkyl)(C₁ to C₆ alkyl), N(C₁ to C₆ alkyl)(C₂ to C₆ alkenyl), and N(C₁ to C₆ alkyl)(heterocycle); or q is 2 and two R³ groups are joined to form an optionally substituted 6-membered aryl, optionally substituted 5- or 6-membered carbocyclic ring, or optionally substituted 5- or 6-membered heterocycle or heteroaryl containing 1 to 3 oxygen, nitrogen, or sulfur atoms and 4 or 5 carbon atoms; m is 1 to 5; n is 1 to 3; p is 0 to 2; q is 0 to 4; and X— is a halogen ion, trifluoroacetate, sulfate, phosphate, acetate, fumarate, maleate, citrate, pyruvate, succinate, oxalate, bisulfate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, D-mandelate, L-mandelate, propionate, tartarate, phthalate, hydrochlorate, hydrobromate, nitrate, methanesulfonate, ethanesulfonate, napthalenesulfonate, benzenesulfonate, toluenesulfonate, mesitylenesulfonate, camphorsulfonate or trifluoromethanesulfonate. In an embodiment, the compound of Formula (III) is:

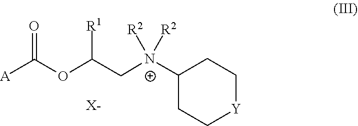

wherein, R¹ is H or C₁ to C₆ alkyl. In one embodiment, R¹ is H. In another embodiment, R¹ is CH₃. R² in Formula (III) is C₁ to C₆ alkyl. In one embodiment, R² is CH₃. In a further embodiment, R² is CH₂CH₃. In yet another embodiment, the two R² groups are joined together to form a 5- or 6-membered ring. The Y substituent is O or CHR³. In one embodiment, Y is O. In another embodiment, Y is CHR³. The R³ moiety is H or C₁ to C₆ alkyl. In one embodiment, R³ is H. In a further embodiment, R³ is CH₃. A of Formula (III) is optionally substituted phenyl, optionally substituted heteroaryl or optionally substituted cycloalkyl. However, when A is unsubstituted phenyl, R¹ and R² are not methyl and R³ is not H. X is chloride, bromide, iodide, trifluoroacetate, sulfate, phosphate, acetate, fumarate, maleate, citrate, pyruvate, succinate, oxalate, sulfonate, bisulfate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, D-mandelate, L-mandelate, propionate, tartrate, phthalate, hydrochlorate, hydrobromate, nitrate, methanesulfonate, napthalenesulfonate, benzenesulfonate, toluenesulfonate, camphorsulfonate or trifluoromethanesulfonate.

In an embodiment, the formulation further comprises a TRPV1 receptor activator.

In an aspect, methods for treating inflammation, pruritus and/or pain, or for treating conditions for which the signs and symptoms include inflammation, pruritis and/or pain, are provided and comprise administering a topical pharmaceutical formulation comprising a poloxamer and a therapeutically effective amount of a compound selected from the group consisting of Formula (I), Formula (II), and Formula (III), or a pharmaceutically acceptable salt or prodrug thereof, to a patient in need thereof. In one embodiment, the methods also comprise administration of a TRPV1 receptor activator.

In an aspect, use of an active ingredient and a hydrophilic non-ionic surfactant for preparation of a medicament for treating inflammation, pruritus and/or pain, or for treating conditions for which the signs and symptoms include inflammation, pruritis and/or pain, are provided, wherein the medicament is a topical formulation comprising a poloxamer and a therapeutically effective amount of a compound selected from the group consisting of Formula (I), Formula (II), and Formula (III), or a pharmaceutically acceptable salt or prodrug thereof. In one embodiment, the medicament further comprises a TRPV1 receptor activator. The medicament can be administered by a dosing regimen comprising topically administering the medicament to a subject in need thereof.

In an aspect, the formulations for treating inflammation, pruritus and/or pain, and for treating conditions for which the signs and symptoms include inflammation, pruritis and/or pain, are provided wherein the pruritis and/or pain, may include, but is not limited to, that associated with atopic dermatitis, eczema (including hand and foot eczema such as chronic hand eczema), urticaria, psoriasis and other acute and chronic itch conditions, chronic pain, neuropathic pain, somatic pain, idiopathic pain, dysfunctional pain, nociceptive pain, neuropathic pain, inflammatory pain, procedural pain, or migraine.

In an aspect, methods for manufacturing topical pharmaceutical formulations are provided comprising the steps of making a first mixture by dissolving an active ingredient comprising a compound selected from the group consisting of Formula (I), Formula (II), and Formula (III) in a combination of propylene glycol and water; making a second mixture by combining a hydrophilic non-ionic surfactant comprising a poloxamer with water; and mixing the first mixture and the second mixture to form a homogenous clear gel.

In another aspect, the present disclosure provides a dosage form comprising a pharmaceutical formulation comprising the active ingredient as disclosed herein and one or more stabilizing polymers, wherein the pharmaceutical formulation remains in substantially gel-like form after storage of the pharmaceutical formulation for a predetermined time and under predetermined conditions.

In still another aspect, methods for inhibiting sodium channel response by a compound selected from the group consisting of Formula (I), Formula (II), and Formula (III) are provided, comprising administering to a subject a topical pharmaceutical formulation of the disclosure.

In another aspect, the present disclosure provides a method of manufacturing or stabilizing a pharmaceutical formulation, comprising the steps of mixing an active ingredient comprising a compound selected from the group consisting of Formula (I), Formula (II), and Formula (III), and one or more hydrophilic non-ionic surfactants comprising a poloxamer.

In another aspect, the present disclosure provides a kit comprising one or more dosage forms and instructions for administering the dosage forms to a subject, wherein the dosage forms comprise a pharmaceutical formulation comprising an active ingredient and one or more hydrophilic non-ionic surfactants, wherein the active ingredient comprises a compound selected from the group consisting of Formula (I), Formula (II), and Formula (III), wherein the dosage form remains in substantially gel-like form after storage of the pharmaceutical formulation for a predetermined time and under predetermined conditions. The kit may further comprise administration instructions.

### BRIEF DESCRIPTION OF THE DRAWINGS

FIG. 1A is bar graph and FIG. 1B is scatter dot plot showing the total number of scratches in a 40 minutes period following CQ injection 6 hours after the application of HEC gel (Vehicle control) or HEC+COMPOUND A (3%), Poloxamer gel (Veh control) or Poloxamer+COMPOUND A (3%), and ointment (Veh control) or Ointment+COMPOUND A (3%) to mice.

FIG. 2A is bar graph and FIG. 2B is a scatter dot plot showing the total number of scratches in the 40 min period following CQ injection in Naïve mice (no test article or vehicle) and in mice 6 hrs after the application of Gel+COMPOUND A 0.3%, 1%, 3% and 5%; DMSO+COMPOUND A 3% and 5% Gel+COMPOUND A, DMSO+COMPOUND A; and Gel or DMSO vehicle controls.

FIGS. 3A and 3B are line graphs showing, respectively, scratches/min following CQ treatment for the test article (Gel+COMPOUND A 0.3%, 1%, 3% and 5%) and DMSO (DMSO+COMPOUND A 3% and 5%) treatment groups.

FIG. 4A is a bar graph and FIG. 4B is scatter dot plot that represents the pooled data from Phases 1 and 2 showing the total number of scratches following CQ injection over 40 min period on day 5 in groups of mice (n=4 each) that received placebo (“Polax Gel+CQ”), poloxamer gel with 0.1%, 0.3%, 1%, 3% COMPOUND A, and poloxamer gel with 3% COMPOUND A that was removed 3 hours after application.

FIG. 5A is a bar graph and FIG. 5B is scatter dot plot that represents the total number of scratches following CQ injection over 40 min period on day 5 in groups of mice (n=8 each) that received poloxamer gel with 0.1%, 0.3%, 1%, 3% COMPOUND A, and Poloxamer gel with 3% COMPOUND A applied 72 hrs after shaving the injection site (indicated by #), placebo (“Polax Gel+CQ”) and placebo without CQ (“Polax Gel”).

FIG. 6A is a bar graph and FIG. 6B is a scatter dot plot showing the total number of scratches in the 40 min period following CQ injection 3 hrs, 6 hrs, and 15 hrs after the application of Gel+COMPOUND A (1%, 2% and 3%) or Gel Vehicle controls.

FIG. 7 is a bar graph showing the total number of scratches in the 40 min period following CQ injection at 3 hrs, 6 hrs, and 15 hrs after the application of Gel+COMPOUND A 3% (3% ASANA) or Gel alone (Gel), with or without its removal at 3 hrs.

FIG. 8 is a bar graph showing the total number of scratches at 1, 3, 6, 15 and 24 hours after administration of COMPOUND A 3% poloxamer gel (COMPOUND A (3%)) and placebo gel (Polax gel).

### DETAILED DESCRIPTION

The following detailed description is exemplary and explanatory and is intended to provide further explanation of the present disclosure described herein. Other advantages, and novel features will be readily apparent to one of ordinary skill in the art from the following detailed description of the present disclosure.

The disclosure provides one or more topical pharmaceutical formulations comprising an active ingredient and a poloxamer, methods of manufacturing, kits, methods of treating, and dosage forms. The active ingredient is configured to regulate sodium channel activity such that the pharmaceutical formulations are capable of treating conditions associated with sodium channel activity, including, for example, inflammation, pruritus and/or pain when administered topically.

In one embodiment, a pharmaceutical formulation of the disclosure comprises an active ingredient and a hydrophilic non-ionic surfactant comprising a poloxamer, wherein the pharmaceutical formulation is in substantially gel-like form and the active ingredient comprises a compound selected from the group consisting of Formula (I), Formula (II), and Formula (III). In one embodiment, Formula (I) and (II) are:

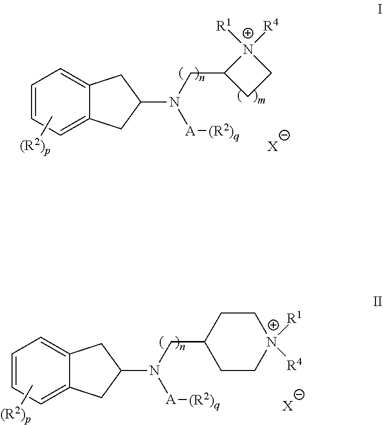

wherein: A is phenyl or heteroaryl; R¹ and R⁴ are, independently, C₁ to C₆ alkyl or CH₂CH₂OH; or R¹ and R⁴ are joined to form a 4- or 6-membered carbocyclic or heterocyclic ring; R² is independently selected from the group consisting of hydrogen, halogen, NO₂, OH, and C₁ to C₆ alkoxy; R³ is independently selected from the group consisting of hydrogen, halogen, CN, NO₂, NH₂, optionally substituted C₁ to C₆ alkyl, C₂ to C₆ alkenyl, C₂ to C₆ alkynyl, OH, CF₃, OCF₃, SCF₃, optionally substituted C₁ to C₆ alkoxy, C₂ to C₆ alkynyloxy, heterocyclyloxy, heteroaryloxy, optionally substituted C₁ to C₆ alkylthio, heteroarylthio, C(O)O(C₁ to C₆ alkyl), C(O)(C₁ to C₆ alkyl), C(O)(aryl), C(O)(heterocycle), C(O)NH₂, C(O)NH(C₁ to C₆ alkyl), C(O)NH(aryl), C(O)NH(heterocycle), C(O)NH(heteroaryl), C(O)N(C₁ to C₆ alkyl)(C₁ to C₆ alkyl), C(O)N(aryl)(C₁ to C₆ alkyl), C(S)NH₂, optionally substituted aryl, heteroaryl, heterocycle, NHC(O)(C₁ to C₆ alkyl), NHC(O)(aryl), NHC(O)(heteroaryl), NHC(O)O(C₁ to C₆ alkyl), N(C₁ to C₆ alkyl)C(O)(C₁ to C₆ alkyl), N(C₁ to C₆ alkyl)C(O)O(C₁ to C₆ alkyl), NHC(O)NH₂, NHC(O)NH(C₁ to C₆ alkyl), NHC(O)NH(heteroaryl), NHSO₂(C₁ to C₆ alkyl), SO₂(C₁ to C₆ alkyl), SO₂NH₂, SO₂NH(C₁ to C₆ alkyl), SO₂NH(C₂ to C₆ alkynyl), SO₂N(C₁ to C₆ alkyl)(C₁ to C₆ alkyl), SO₂NH(heteroaryl), NH(C₁ to C₆ alkyl), N(C₁ to C₆ alkyl)(C₁ to C₆ alkyl), N(C₁ to C₆ alkyl)(C₂ to C₆ alkenyl), and N(C₁ to C₆ alkyl)(heterocycle); or q is 2 and two R³ groups are joined to form an optionally substituted 6-membered aryl, optionally substituted 5- or 6-membered carbocyclic ring, or optionally substituted 5- or 6-membered heterocycle or heteroaryl containing 1 to 3 oxygen, nitrogen, or sulfur atoms and 4 or 5 carbon atoms; m is 1 to 5; n is 1 to 3; p is 0 to 2; q is 0 to 4; and X⁻ is a halogen ion, trifluoroacetate, sulfate, phosphate, acetate, fumarate, maleate, citrate, pyruvate, succinate, oxalate, bisulfate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, D-mandelate, L-mandelate, propionate, tartarate, phthalate, hydrochlorate, hydrobromate, nitrate, methanesulfonate, ethanesulfonate, napthalenesulfonate, benzenesulfonate, toluenesulfonate, mesitylenesulfonate, camphorsulfonate or trifluoromethanesulfonate.

In one embodiment, the compound of Formula (III) is:

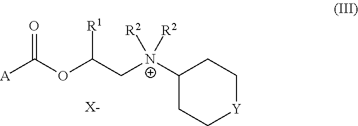

wherein, R¹ is H or C₁ to C₆ alkyl. In one embodiment, R¹ is H. In another embodiment, R¹ is CH₃. R² in Formula (III) is C₁ to C₆ alkyl. In one embodiment, R² is CH₃. In a further embodiment, R² is CH₂CH₃. In yet another embodiment, the two R² groups are joined together to form a 5- or 6-membered ring. The Y substituent is O or CHR³. In one embodiment, Y is O. In another embodiment, Y is CHR³. The R³ moiety is H or C₁ to C₆ alkyl. In one embodiment, R³ is H. In a further embodiment, R³ is CH₃. A of Formula (III) is optionally substituted phenyl, optionally substituted heteroaryl or optionally substituted cycloalkyl. However, when A is unsubstituted phenyl, R¹ and R² are not methyl and R³ is not H. In some embodiments, Formula (III) can be defined as follows:
i. In one embodiment, A is:

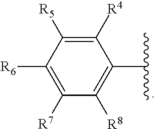

In this structure, R⁴, R⁵, R⁶, R⁷, and R⁸ are, independently, selected from among H, optionally substituted C₁ to C₆ alkyl, optionally substituted C₁ to C₆ alkoxy, halogen, C₁ to C₃ perfluoroalkyl, and NO₂.
ii. In another embodiment, A is:

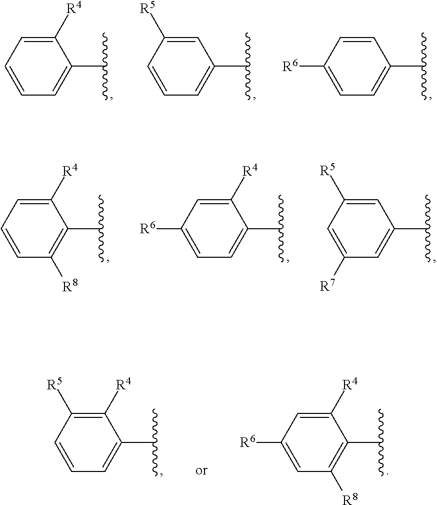

iii. In a further embodiment, A is of the structures noted in options i or ii and R⁴, R⁵, R⁶, R⁷, and R⁸ are, independently, selected from among OCH₃, CH₃, CH₂CH₃, CH(CH₃)₂, C(CH₃)₃, Cl, F, CF₃, and NO₂.
iv. In still another embodiment, A is an optionally substituted pyrrole.
v. In yet a further embodiment, A is:

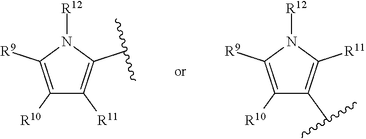

In these structures, R⁹, R¹⁰, and R¹¹ are, independently, H, optionally substituted C₁ to C₆ alkyl, or halogen; and R¹² is H or C₁ to C₆ alkyl.
vi. In another embodiment, A is the following and R¹² is defined as noted in option v:

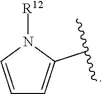

vii. In still a further embodiment, R¹² is CH₃ in options v or vi noted above.
viii. In yet a further embodiment, A is an optionally substituted thiophene.
ix. In another embodiment, A is:

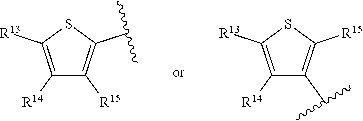

In these structures, R¹³, R¹⁴, and R¹⁵ are, independently, H, optionally substituted C₁ to C₆ alkyl, or halogen.
x. In still another embodiment, A is:

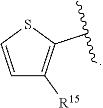

xi. In yet a further embodiment, R¹⁵ in options ix or x noted above is CH₃.
xii. In another embodiment, A is an optionally substituted benzothiophene.
xiii. In still another embodiment, A is:

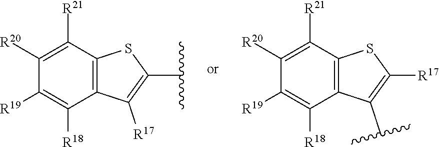

In these structures, R¹⁷, R¹⁸, R¹⁹, R²⁰, and R²¹ are, independently, H, optionally substituted C₁ to C₆ alkyl, or halogen.
xiv. In a further embodiment, A is of the following structure and R¹⁷ is defined as in option xiii:

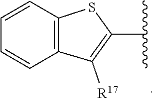

xv. In yet another embodiment, R¹⁷ as in options xiii and xiv is halogen.

In the compounds of Formula (III), X⁻ can be chloride, bromide, iodide, trifluoroacetate, sulfate, phosphate, acetate, fumarate, maleate, citrate, pyruvate, succinate, oxalate, sulfonate, bisulfate, malonate, xinafoate, ascorbate, oleate, nicotinate, saccharinate, adipate, formate, glycolate, L-lactate, D-lactate, aspartate, malate, tartrate, L-tartrate, D-tartrate, stearate, 2-furoate, 3-furoate, napadisylate, edisylate, isethionate, D-mandelate, L-mandelate, propionate, phthalate, hydrochlorate, hydrobromate, nitrate, methanesulfonate, napthalenesulfonate, benzenesulfonate, toluenesulfonate, camphorsulfonate or trifluoromethanesulfonate.

The following definitions are used in connection with the compounds described herein. In general, the number of carbon atoms present in a given group is designated “Cₓ to C_{y}”, where x and y are the lower and upper limits, respectively. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are determined by naming from left to right the terminal portion of the functionality followed by the adjacent functionality toward the point of attachment. As used herein, “optionally substituted” means that at least 1 hydrogen atom of the optionally substituted group has been replaced.

“Alkyl” refers to a hydrocarbon chain that may be straight or branched. In one embodiment, an alkyl contains 1 to 6 (inclusive) carbon atoms. In another embodiment, an alkyl contains 1 to 5 (inclusive) carbon atoms. In a further embodiment, an alkyl contains 1 to 4 (inclusive) carbon atoms. In yet another embodiment, an alkyl contains 1 to 3 (inclusive) carbon atoms. In still a further embodiment, an alkyl contains 1 or 2 carbon atoms. Examples of alkyl groups that are hydrocarbon chains include, but are not limited to, methyl, ethyl, propyl, butyl, pentyl, and hexyl, where all isomers of these examples are contemplated.

Alkyl groups may also consist of or contain a cyclic alkyl radical, i.e., “carbocyclic ring”. Examples of carbocyclic rings include, but are not limited to, cyclopropyl, cyclobutyl, cyclopentyl, cyclohexyl, and the like. In one embodiment, the carbocyclic ring is 3- to 6-membered. In a further embodiment, the carbocyclic ring is 3- to 5-membered. In still a further embodiment, the carbocyclic ring is 4- to 6-membered. In another embodiment, the carbocyclic ring is 3- or 4-membered, i.e., cyclopropyl or cyclobutyl. Unless specifically noted, the alkyl groups are unsubstituted, i.e., they contain carbon and hydrogen atoms only. However, when the alkyl group or carbocyclic ring is substituted, it is prefaced with the term “optionally substituted” or “substituted”. The optional substituents of the alkyl groups or carbocyclic rings include, without limitation, halogen, CN, NO₂, C₁ to C₆ alkyl, OH, C₁ to C₆ alkoxy, C₁ to C₆ alkoxy-C₁ to C₆ alkoxy, C₁ to C₆ alkoxy-C₁ to C₆ alkyl-C₁ to C₆ alkoxy, heterocyclyloxy, C₁ to C₆ alkylthio, aryl, heterocycle, heteroaryl, C(O)(C₁ to C₆ alkyl), C(O)(heterocycle), C(O)O(C₁ to C₆ alkyl), C(O)NH₂, C(O)NH(C₁ to C₆ alkyl), C(O)N(C₁ to C₆ alkyl)(C₁ to C₆ alkyl), SO₂(C₁ to C₆ alkyl), SO₂(C₂ to C₆ alkynyl), SO₂NH(C₁ to C₆ alkyl), SO₂(heterocycle), NHC(O)(C₁ to C₆ alkyl), NHSO₂(C₁ to C₆ alkyl), N(C₁ to C₆ alkyl)SO₂(C₁ to C₆ alkyl), NH₂, NH(aryl), N(C₁ to C₆ alkyl)(C₁ to C₆ alkyl), or NHC(O)NH₂.

“Alkoxy” refers to O(alkyl), where the alkyl is optionally substituted and is defined above. In one embodiment, an alkoxy contains 1 to 6 (inclusive) carbon atoms or integers or ranges there between. In another embodiment, an alkoxy contains 1 to 5 (inclusive) carbon atoms or ranges there between. In a further embodiment, an alkoxy contains 1 to 4 (inclusive) carbon atoms. In yet another embodiment, an alkoxy contains 1 to 3 (inclusive) carbon atoms. In still a further embodiment, an alkoxy contains 1 or 2 carbon atoms. Examples of alkoxy include, but are not limited to, methoxy, ethoxy, propoxy, and butoxy. The alkyl radical of an alkoxy group can be unsubstituted or substituted as defined above for “alkyl”.

“Aryl” refers to an aromatic hydrocarbon group containing carbon atoms. In one embodiment, the aryl contains 6 to 10 carbon atoms, i.e., 6-, 7-, 8-, 9- or 10-membered. In another embodiment, aryl is an aromatic or partly aromatic bicyclic group. In a further embodiment, the aryl is a phenyl group. In another embodiment, the aryl is naphthyl (such as α-naphthyl or β-naphthyl), 1,2,3,4-tetrahydronaphthyl, or indanyl. An aryl group can be unsubstituted or substituted with one or more groups including, without limitation, halogen, NO₂, C₁ to C₆ alkyl, OH, C₁ to C₆ alkoxy, C₁ to C₆ alkoxy-C₁ to C₆ alkoxy, C₁ to C₆ alkoxy-C₁ to C₆ alkoxy-C₁ to C₆ alkoxy, heterocyclyloxy, C₁ to C₆ alkylthio, aryl, heterocycle, heteroaryl, C(O)(C₁ to C₆ alkyl), C(O)(heterocycle), C(O)O(C₁ to C₆ alkyl), C(O)NH₂, C(O)NH(C₁ to C₆ alkyl), C(O)N(C₁ to C₆ alkyl)(C₁ to C₆ alkyl), SO₂(C₁ to C₆ alkyl), SO₂(C₂ to C₆ alkynyl), SO₂NH(C₁ to C₆ alkyl), SO₂(heterocycle), NHSO₂(C₁ to C₆ alkyl), N(C₁ to C₆ alkyl)SO₂(C₁ to C₆ alkyl), NH₂, NH(aryl) or NHC(O)NH₂.

“Halogen” refers to F, Cl, Br and I and “halogen ion” refers to their ionized forms (for example chloride, bromide or iodide).

The term “heteroatom” refers to a sulfur, nitrogen, or oxygen atom.

“Heteroaryl” refers to a monocyclic aromatic 5- or 6-membered ring containing at least one ring heteroatom. In one embodiment, the heteroaryl contains 1 to 5 carbon atoms (inclusive) or integers or ranges there between. In a further embodiment, the heteroaryl contains 2 to 5 carbon atoms (inclusive). In another embodiment, the heteroaryl contains 3 to 5 carbon atoms (inclusive). In still a further embodiment, the heteroaryl contains 4 or 5 carbon atoms. “Heteroaryl” also refers to bicyclic aromatic ring systems wherein a heteroaryl group as just described is fused to at least one other cyclic moiety. In one embodiment, a phenyl radical is fused to a 5- or 6-membered monocyclic heteroaryl to form the bicyclic heteroaryl. In another embodiment, a cyclic alkyl is fused to a monocyclic heteroaryl to form the bicyclic heteroaryl. In yet a further embodiment, the bicyclic heteroaryl is a pyridine fused to a 5- or 6-membered monocyclic heteroaryl. In still another embodiment, the heteroaryl ring has 1 or 2 nitrogen atoms in the ring. In a further embodiment, the heteroaryl ring has 1 nitrogen atom and 1 oxygen atom. In yet another embodiment, the heteroaryl ring has 1 nitrogen atom and 1 sulfur atom. Examples of heteroaryl groups include, without limitation, furan, thiophene, indole, azaindole, oxazole, thiazole, isoxazole, isothiazole, imidazole, pyridine, pyrimidine, pyrazine, pyridazine, pyrrole, 1,3,4-oxadiazole, 1,2,4-triazole, tetrazole, benzoxazole, benzothiazole, benzofuran, benzisoxazole, benzimidazole, azabenzimidazole, indazole, quinazoline, quinoline, and isoquinoline. A heteroaryl may be unsubstituted or substituted with one or more groups including, without limitation, halogen, CN, NO₂, C₁ to C₆ alkyl, OH, C₁ to C₆ alkoxy, C₁ to C₆ alkoxy-C₁ to C₆ alkoxy, C₁ to C₆ alkoxy-C₁ to C₆ alkoxy-C₁ to C₆ alkoxy, heterocyclyloxy, C₁ to C₆ alkylthio, aryl, heterocycle, heteroaryl, C(O)(C₁ to C₆ alkyl), C(O)(heterocycle), C(O)O(C₁ to C₆ alkyl), C(O)NH₂, C(O)NH(C₁ to C₆ alkyl), C(O)N(C₁ to C₆ alkyl)(C₁ to C₆ alkyl), SO₂(C₁ to C₆ alkyl), SO₂(C₂ to C₆ alkynyl), SO₂NH(C₁ to C₆ alkyl), SO₂(heterocycle), NHC(O)(C₁ to C₆ alkyl), NHSO₂(C₁ to C₆ alkyl), N(C₁ to C₆ alkyl)SO₂(C₁ to C₆ alkyl), NH₂, NH(aryl), N(C₁ to C₆ alkyl)(C₁ to C₆ alkyl) or NHC(O)NH₂.

“Heterocycle” refers to a monocyclic or bicyclic group in which at least 1 ring atom is a heteroatom. A heterocycle may be saturated or partially saturated. In one embodiment, the heterocycle contains 3 to 7 carbon atoms (inclusive) or integers or ranges there between. In a further embodiment, the heterocycle contains 4 to 7 carbon atoms (inclusive). In another embodiment, the heterocycle contains 4 to 6 carbon atoms (inclusive). In still a further embodiment, the heterocycle contains 5 or 6 carbon atoms (inclusive). Examples of heterocycles include, but are not limited, to aziridine, oxirane, thiirane, morpholine, thiomorpholine, pyrroline, pyrrolidine, azepane, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, homopiperazine, oxazine, azecane, tetrahydroquinoline, perhydroisoquinoline, 5,6-dihydro-4H-1,3-oxazin-2-yl, 2,5-diazabicyclo[2.2.1]heptane, 2,5-diazabicyclo[2.2.2]octane, 3,6-diazabicyclo[3.1.1]heptane, 3,8-diazabicyclo[3.2.1]octane, 6-oxa-3,8-diazabicyclo[3.2.1]octane, 7-oxa-2,5-diazabicyclo[2.2.2]octane, 2,7-dioxa-5-azabicyclo[2.2.2]octane, 2-oxa-5-azabicyclo[2.2.1]heptane-5-yl, 2-oxa-5-azabicyclo[2.2.2]octane, 3,6-dioxa-8-azabicyclo[3.2.1]octane, 3-oxa-6-azabicyclo[3.1.1]heptane, 3-oxa-8-azabicyclo[3.2.1]octan-8-yl, 5,7-dioxa-2-azabicyclo[2.2.2]octane, 6,8-dioxa-3-azabicyclo[3.2.1]octane, 6-oxa-3-azabicyclo[3.1.1]heptane, 8-oxa-3-azabicyclo[3.2.1]octan-3-yl, 2,5-diazabicyclo[2.2.1]heptane-5-yl, 6-azabicyclo[3.2.1]oct-6-yl, 8-azabicyclo[3.2.1]octan-8-yl, 3-oxa-7,9-diazabicyclo[3.3.1]nonan-9-yl, 9-oxa-3-azabicyclo[3.3.1]nonan-3-yl, 3-oxa-9-azabicyclo[3.3.1]nonan-9-yl, 3,7-dioxa-9-azabicyclo[3.3.1]nonan-9-yl, 3,4-dihydro-2H-1,4-benzoxazin-7-yl, thiazin, dithiane, and dioxane. In another embodiment, the heterocycle contains 1 or 2 nitrogen atoms. In a further embodiment, the heterocycle contains 1 or 2 nitrogen atoms and 3 to 6 carbon atoms. In yet another embodiment, the heterocycle contains 1 or 2 nitrogen atoms, 3 to 6 carbon atoms, and 1 oxygen atom. In a further embodiment, the heterocycle is 5- to 8-membered. In another embodiment, the heterocycle is 5-membered. In still a further embodiment, the heterocycle is 6-membered. In yet another embodiment, the heterocycle is 8-membered. A heterocycle may be unsubstituted or substituted with one or more groups including, without limitation, halogen, CN, NO₂, C₁ to C₆ alkyl, OH, C₁ to C₆ alkoxy, C₁ to C₆ alkoxy-C₁ to C₆ alkoxy, C₁ to C₆ alkoxy-C₁ to C₆ alkoxy-C₁ to C₆ alkoxy, heterocyclyloxy, C₁ to C₆ alkylthio, aryl, heterocycle, heteroaryl, C(O)(C₁ to C₆ alkyl), C(O)(heterocycle), C(O)O(C₁ to C₆ alkyl), C(O)NH₂, C(O)NH(C₁ to C₆ alkyl), C(O)N(C₁ to C₆ alkyl)(C₁ to C₆ alkyl), SO₂(C₁ to C₆ alkyl), SO₂(C₂ to C₆ alkynyl), SO₂NH(C₁ to C₆ alkyl), SO₂(heterocycle), NHC(O)(C₁ to C₆ alkyl), NHSO₂(C₁ to C₆ alkyl), N(C₁ to C₆ alkyl)SO₂(C₁ to C₆ alkyl), NH₂, NH(aryl), N(C₁ to C₆ alkyl)(C₁ to C₆ alkyl) or NHC(O)NH₂.

“Alkylamino” refers to an NH or N group, the nitrogen atom of the group being attached to 1 or 2 alkyl substituents, respectively, wherein the alkyl is optionally substituted and defined above. The alkylamino is bound through the nitrogen atom of the group. In one embodiment, alkylamino refers to NH(alkyl). In another embodiment, alkylamino refers to N(alkyl)(alkyl), i.e., a “dialkylamino”. In a further embodiment, alkylamino refers to N(C₁ to C₆ alkyl)(C₁ to C₆ alkyl). In yet another embodiment, alkylamino refers to N(alkyl)(heterocycle). In still a further embodiment, alkylamino refers to N(alkyl)(aryl). In another embodiment, alkylamino refers to N(alkyl)(heteroaryl). In et a further embodiment, alkylamino refers to N(alkyl)(alkenyl). When the nitrogen atom is bound to two alkyl groups, each alkyl group may be independently selected. In another embodiment, two alkyl groups on the nitrogen atom may be taken together with the nitrogen to which they are attached to forma 3- to 4-membered nitrogen-containing heterocycle where up to two of the carbon atoms of the heterocycle can be replaced with N(H), N(C₁ to C₆ alkyl), N(aryl), N(heteroaryl), O, S(O), or S(O)₂. Examples of alkylamino include, but are not limited to N(CH₃)₂, N(CH₂CH₃)(CH₃), N(CH₂CH₃)₂, N(CH₂CH₂CH₃)₂, N(CH₂CH₂CH₂CH₃)₂, N(CH(CH₃)₂)(CH₃), and the like.

“Arylamino” refers to an NH or N group, the nitrogen atom of the group being attached to 1 or 2 aryl substituents, respectively, wherein the aryl is optionally substituted and defined above. The arylamino is bound through the nitrogen atom of the group. In one embodiment, arylamino refers to NH(aryl). In another embodiment, arylamino refers to N(aryl)(aryl), i.e., a “diarylamino”. When the nitrogen atom is bound to two aryl groups, each aryl may be independently selected.

“Alkylcarbonylamino” refers to NHC(O)(alkyl) or N(alkyl)C(O)(alkyl) where the alkyl groups are independently defined and independently optionally substituted as described above. Examples of alkylcarbonylamino include, but are not limited to, CH₃CONH, CH₃CH₂CONH, CH₃CH₂CH₂CONH, CH₃CH(CH₃)CONH, and the like.

“Ester” refers to C(O)O(alkyl), which is bound through the carbon atom. The alkyl group is defined and optionally substituted as described above. Examples of ester include, without limitation, C(O)OCH₃, C(O)O(CH₂CH₃), C(O)O(CH₂CH₂CH₃), C(O)(O)(CH₂CH₂CH₂CH₃), and the like.

“Urea” refers to a group having a NHC(O)NH where one of the nitrogen atoms is bound to an alkyl or heteroaryl group. The alkyl or heteroaryl groups are defined and optionally substituted as described above. Examples of urea include, without limitation, NHC(O)NHCH₃, NHC(O)NHCH₂CH₃, NHC(O)NHCH₂CH₂CH₃, NHC(O)NHCH₂CH₂CH₂CH₃, and the like.

“Alkylaminocarbonyl” refers to C(O)NH(alkyl) or C(O)N(alkyl)(alkyl) where the alkyl groups are independently defined and independently optionally substituted as described above. Examples of alkylaminocarbonyl include, but are not limited to, CH₃NHCO, CH₃CH₂NHCO, CH₃CH₂CH₂NHCO, CH₃CH(CH₃)NHCO, and the like.

A “patient” or “subject” is a mammal, e.g., a human or a veterinary patient or subject, e.g., mouse, rat, guinea pig, dog, cat, horse, cow, pig, or non-human primate, such as a monkey, chimpanzee, baboon or gorilla.

The terms “comprise”, “comprises”, and “comprising” are to be interpreted inclusively rather than exclusively. The terms “consist”, “consisting”, and its variants, are to be interpreted exclusively, rather than inclusively.

As used herein, the term “about” means a variability of 10% from the reference given, unless otherwise specified.

As used herein, an “active moiety” of the disclosure means an active ingredient as disclosed herein without X.

“Methanesulfonate” is also known in the art as “mesylate”.

Compounds of Formulas (I), (II) and (III) possess one or more chiral centers, and it is specifically contemplated that each separate enantiomer of compounds comprising an active ingredient of the disclosure, as well as mixtures of the enantiomers, can be used in the present formulations and methods. As disclosed herein, all chiral, enantiomeric and racemic forms of a chemical structure are intended, unless the specific stereochemistry is indicated. It is well known in the art how to prepare optically active forms of the compounds comprising active ingredients of the present formulations and methods, such as by resolution of racemic forms or by synthesis from optically active starting materials.

In another embodiment, the active ingredient comprises (R)-2-(2-((2,3-dihydro-1H-inden-2-yl)(o-tolyl)amino)ethyl)-1,1-dimethylpiperidin-1-ium (sometimes referred to herein and in the Figures as “COMPOUND A” or “CMPD A”), or pharmaceutically acceptable salts thereof. In some embodiments, the pharmaceutically acceptable salt of COMPOUND A is the bromide salt:

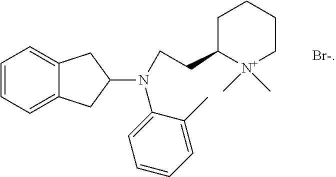

In some embodiments, the pharmaceutically acceptable salt of COMPOUND A is the sulfate salt. In some embodiments, the pharmaceutically acceptable salt of COMPOUND A is the methanesulfonate salt.

In some embodiments, the active ingredient comprises (R)—N-[2-((4-(tert-butyl)benzoyl)oxy)propyl]-N,N-dimethylcyclohexanaminium bromide:

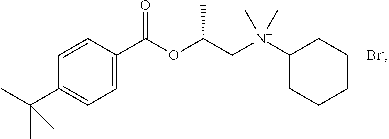 [SMILES: C[C@H](C[N+](C)(C)C1CCCCC1)OC(=O)c1ccc(C(C)(C)C)cc1]

or pharmaceutically acceptable salts thereof. In some embodiments, the pharmaceutically acceptable salt is the bromide salt. In some embodiments, the pharmaceutically acceptable salt is the sulfate salt. In some embodiments, the pharmaceutically acceptable salt is the methanesulfonate salt.

In certain embodiments, the active ingredient comprises about 0.3% to about 7.5% w/w of the formulation; for example about 0.3%, 1% w/w, about 3% w/w of the formulation, about 5% w/w of the formulation, or about 7% w/w of the formulation, about 7.5% w/w of the formulation. In certain embodiments, the active ingredient comprises about 0.05% to about 10% w/w of the formulation; for example about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0% w/w of the formulation. In certain embodiments, the active ingredient comprises about 0.1% to about 1.0% w/w of the formulation; for example about 0.1%, about 0.3%, about 0.5%, about 0.7% w/w of the formulation, or about 1% w/w of the formulation. In one embodiment, the active ingredient comprises about 1.0% w/w of the formulation of the active ingredient, which is equivalent to 0.82% w/w of the formulation of the active moiety; or about 3.0% w/w of the formulation of the active ingredient, which is equivalent to about 2.46% w/w of the formulation of the active moiety.

The poloxamer may comprise any suitable poloxamer, for example Pluronic®, Synperonic® or Kolliphor® P407. In an embodiment, the poloxamer is Kolliphor® P407 and comprises above about 22% w/w, for example about 15 to about 40% w/w of the pharmaceutical formulation, about 20 to about 30% w/w of the pharmaceutical formulation, or about 15%, 20%, 25% or 30% w/w of the pharmaceutical formulation. In certain embodiments, the pharmaceutical formulation further comprises water and one or more solvents, and the one or more solvents may comprise propylene glycol. In some embodiments, water comprises about 40 to about 50% w/w of the formulation, and the propylene glycol comprises about 15 to 25% w/w of the formulation. In other embodiments, water comprises about 42, about 47, or about 49% w/w of the formulation, and the propylene glycol comprises about 20% of the formulation.

The pharmaceutical formulations disclosed herein may comprise topical compositions having improved stability profiles. The compositions may be suitable for topical delivery, for example transdermal or transmucosal delivery. In an embodiment, substantially no discoloration and substantially no decrease in viscosity of the pharmaceutical formulation occurs at storage conditions comprising 40° C. and 75% relative humidity. The formulations are stable, with respect to discoloration and viscosity at about 40° C. and about 75% relative humidity (“RH”) for a period of at least 12 months. Furthermore, in an embodiment, following storage of the compositions for 3 months or longer, under standard storage conditions or accelerated conditions, the total amount of impurities present in the compositions is not more than about 3%: standard storage conditions may comprise a temperature of about 20 to 25° C. and no more than about 40% RH.

In an embodiment, administration of the formulation delivers 3-75 mg/mL dose of the active ingredient via one or more of topical delivery routes.

In an embodiment, the formulations of the present disclosure further comprise other ingredients that can be used in a topical pharmaceutical formulation, such as a fragrance. The formulations may be packaged in containers known to one skilled in the art, including but not limited to tubes, transdermal patches, bandages for minor wounds such as Band-aid® brand adhesive bandages, and the like, configured for single or multiple use.

In an embodiment, the formulations of the present disclosure comprise topical compositions wherein the compositions are formulated for transdermal delivery. The formulations comprise active ingredients comprising about 1-7.5% w/w of the composition or about 0.1-1.0% of the composition; a poloxamer comprising about 30% w/w of the composition, the poloxamer comprising Kolliphor P407; propylene glycol comprising about 20% w/w of the composition; and water comprising about 42-50% w/w of the composition, wherein the active ingredient is a compound of Formula (I), (II) or (III) as defined above, or a pharmaceutically acceptable salt or prodrug thereof. In certain embodiments, such topical compositions may be packaged in tubes, transdermal patches, bandages for minor wounds such as Band-aid® brand adhesive bandages, and the like, configured for single or multiple use.

In an embodiment, the formulations of the present disclosure may be utilized for treating inflammation, pruritus and/or pain, and for treating conditions for which the signs and symptoms include inflammation, pruritis and/or pain. In certain embodiments, the pruritus and/or pain, may include but is not limited to pruritus associated with atopic dermatitis, eczema (including hand and foot eczema, such as chronic hand eczema), urticaria, psoriasis and other acute and chronic itch conditions, chronic pain, neuropathic pain, somatic pain, idiopathic pain, dysfunctional pain, nociceptive pain, neuropathic pain, inflammatory pain, procedural pain, or migraine. Though not wishing to be bound by the following theory, it is believed that administration of the formulation minimizes motor impairment and/or administration of the formulation blocks neuronal sodium-channel activity. In an embodiment, the dosing regimen for treating inflammation, pruritus and/or pain comprises application to the affected area of the skin 1 to 6 times daily. In another embodiment, the dosing regimen for treating inflammation, pruritus and/or pain comprises application to the affected area of the skin twice daily.

In an embodiment, the present disclosure comprises methods for manufacturing topical pharmaceutical formulations comprising: making a first mixture by dissolving an active ingredient in a combination of propylene glycol and water; making a second mixture by combining a hydrophilic non-ionic surfactant comprising a poloxamer with water; and mixing the first mixture and the second mixture to form a homogenous clear gel, wherein the active ingredient comprises a compound of Formula (I), (II), or (III) (as defined above) or a pharmaceutically acceptable salt or prodrug thereof. In an embodiment, the first mixture comprises water in an amount of half the total % water, and the second mixture comprises water at a temperature of 4° C. In an embodiment, the active ingredient comprises about 0.3% w/w, 1% w/w, 2% w/w, 3% w/w, 4% w/w, 5% w/w, 6% w/w, 7% w/w or 7.5% w/w of the formulation. In an embodiment, the active ingredient comprises about 0.1% w/w, 0.2% w/w, 0.3% w/w, 0.4% w/w, 0.5% w/w, 0.6% w/w, 0.7% w/w, 0.8% w/w, 0.9% w/w, or 1.0% w/w of the formulation. In an embodiment, the active ingredient comprises about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0% w/w of the formulation. In one embodiment, the active ingredient comprises about 1.0% w/w of the formulation of the active ingredient, which is equivalent to 0.82% w/w of the formulation of the active moiety; or about 3.0% w/w of the formulation of the active ingredient, which is equivalent to about 2.46% w/w of the formulation of the active moiety. In an embodiment, the poloxamer comprises Pluronic®, Synperonic® or Kolliphor® P407 in about 10 to about 50% w/w, about 25 to about 40% w/w or about 30% w/w of the formulation. In certain embodiments, the formulations comprise one or more solvents such as propylene glycol, which may be present in about 10 to 30% w/w, 15 to 25% w/w, or 20% w/w of the formulation. The methods disclosed herein result in pharmaceutical formulations, suitable for topical use, wherein such pharmaceutical formulations display improved stability, with respect to discoloration and viscosity. In certain embodiments, the pharmaceutical formulations of the present disclosure comprise additional excipients such as a fragrance. The formulations may be packaged in tubes, transdermal patches, bandages for minor wounds such as Band-aid® brand adhesive bandages, and the like, configured for single, or multiple use. In an embodiment the present disclosure further comprises methods for stabilizing disclosed pharmaceutical formulations.

In an embodiment, the pharmaceutical formulations of the present disclosure comprise topically administering to a subject in need thereof a pharmaceutical formulation, wherein the pharmaceutical formulation comprises an active ingredient and a hydrophilic non-ionic surfactant comprising a poloxamer, wherein the dosing regimen comprises administering a single application from a pre-filled container on multiple days, for example, on day 1, day 2 or day 3 following diagnosis or identification of the condition or symptoms to be treated, and additional applications thereafter wherein the single application comprises the active ingredient of the pharmaceutical formulation in a concentration of about 0.3% w/w to 7.5% w/w or about 0.1% w/w to 1.0% w/w, wherein the composition provides a therapeutically effective physiological amount of the active ingredient, and wherein the active ingredient is a compound of Formula (I), (II) or (III) (as described above) or a pharmaceutically acceptable salt or prodrug thereof. In an embodiment, the dosing regimen comprises administering a single application comprising the active ingredient of the pharmaceutical formulation in a concentration of about 0.3% w/w, 1% w/w, 2% w/w, 3% w/w, 5% w/w, 6%, 7% w/w or 7.5% w/w of the formulation. In an embodiment, the dosing regimen comprises administering a single application comprising the pharmaceutical formulation in a concentration of about 0.1% w/w, 0.2% w/w, 0.3% w/w, 0.4% w/w, 0.5% w/w, 0.6% w/w, 0.7% w/w, 0.8% w/w, 0.9% w/w, or 1.0% w/w of the formulation. In an embodiment, the dosing regimen comprises administering a single application comprising the pharmaceutical formulation in a concentration of about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0% w/w of the formulation. In an embodiment, the dosing regimen comprises administering a single application comprising the pharmaceutical formulation in a concentration of about 1.0% w/w of the formulation of the active ingredient, which is equivalent to 0.82% w/w of the formulation of the active moiety; or about 3.0% w/w of the formulation of the active ingredient, which is equivalent to about 2.46% w/w of the formulation of the active moiety. In an embodiment, the poloxamer comprises Pluronic®, Synperonic® or Kolliphor® P407 in about 10% w/w to about 50% w/w, 25% w/w to about 40% w/w or about 30% w/w of the formulation. In certain embodiments, the pharmaceutical formulations of the present disclosure can be administered transdermally.

In an embodiment, the present disclosure provides for the use of an active ingredient and a hydrophilic non-ionic surfactant for preparation of a medicament to treat inflammation, pruritus and/or pain, and for treating conditions for which the signs and symptoms include inflammation, pruritis and/or pain, wherein the medicament is administered by a dosing regimen comprising topically administering to a subject in need a pharmaceutical formulation as described herein. In one embodiment, the medicament further comprises a TRPV1 receptor activator. In a further embodiment, the present disclosure provides methods for blocking neuronal sodium-channel activity comprising administration of the pharmaceutical formulations as described herein.

Also provided herein are kits comprising at least one dosage form of the disclosure, for example a topical pharmaceutical formulation, and instructions for administering the at least one dosage form to a subject. The kit can also comprise packaging or a container housing the at least one dosage form of the disclosure, and can also comprise instructions on storage, administration, dosing or the like and/or an insert regarding the active ingredient. The kit can also comprise instructions for monitoring circulating levels of the active ingredient (or metabolites thereof) once administered, and optionally, materials for performing such assays including, e.g., reagents, well plates, containers, markers or labels, and the like. Other suitable components to include in kits of the disclosure will be readily apparent to one of skill in the art, taking into consideration the desired indication, dosing regimen, storage conditions and route of administration.

The present disclosure further comprises pre-filled containers containing a poloxamer gel comprising a pharmaceutical formulation, the formulation comprising an active ingredient; and a hydrophilic non-ionic surfactant comprising a poloxamer, wherein the active ingredient is a compound of Formula (I), (II) or (III) or a pharmaceutically acceptable salt or prodrug thereof. Suitable pre-filled containers will be readily apparent to one of skill in the art, including but not limited to tubes, transdermal patches, bandages for minor wounds such as Band-aid® brand adhesive bandages, and the like configured for single or multiple use.

In certain embodiments, the present disclosure provides methods for stabilizing a pharmaceutical formulation comprising making a first mixture by dissolving an active ingredient in a combination of propylene glycol and water, the water in an amount of half total %, making a second mixture by combining a hydrophilic non-ionic surfactant with water at a temperature of 4° C. to form a clear gel, and mixing the first mixture and the second mixture to form a homogenous clear gel, wherein the active ingredient is a compound of Formula (I), (II) or (III) described herein or a pharmaceutically acceptable salt or prodrug thereof.

In certain embodiments, the present disclosure provides methods for blocking neuronal sodium-channel activity comprising administering to a subject in need thereof a pharmaceutical formulation, the formulation comprising an active ingredient and a hydrophilic non-ionic surfactant comprising a poloxamer, wherein the active ingredient is a compound of Formula (I), (II) or (III) described herein or a pharmaceutically acceptable salt or prodrug thereof.

Active ingredients of the present disclosure can be prepared, for example, according to the methods disclosed in U.S. Pat. No. 8,865,741, the entire disclosure of which is herein incorporated by reference, and U.S. Pat. No. 8,685,418, the entire disclosure of which is herein incorporated by reference. In some embodiments of the disclosure, an active ingredient comprising the pharmaceutical formulation of the disclosure can be present in at least about 0.1% w/w, 0.2% w/w, 0.3% w/w, 0.4% w/w, 0.5% w/w, 0.6% w/w, 0.7% w/w, 0.8% w/w, 0.9% w/w, 1%, 2%, 3%, 4%, 5%, 6%, 7% or 7.5 w/w. In an embodiment, an active ingredient comprising the pharmaceutical formulation of the present disclosure can be present in an amount of about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, or about 10.0% w/w. In an embodiment, an active ingredient comprising the pharmaceutical formulation of the present disclosure can be present in an amount of about 1.0% w/w of the formulation of the active ingredient, which is equivalent to 0.82% w/w of the formulation of the active moiety; or about 3.0% w/w of the formulation of the active ingredient, which is equivalent to about 2.46% w/w of the formulation of the active moiety.

The active ingredient for use in the present formulations and methods is a compound which regulates sodium channel activity. The sodium channel regulatory activity of the active ingredients of the disclosure makes these compounds useful for manufacturing pharmaceutical formulations, which can be used for treating dermatological conditions such as pruritus and/or pain.

It has now been unexpectedly and surprisingly found that the pharmaceutical formulations of the disclosure can be stabilized, for example in substantially gel-like form by combining them with a certain hydrophilic non-ionic surfactant comprising a poloxamer, and the resulting pharmaceutical formulation can be maintained in a substantially gel-like state throughout manufacture and storage without substantial discoloration or substantial loss of viscosity or consistency.

Poloxamers are thermo-reversible polymers. The viscosity of thermo-reversible polymers is reduced in extremes of temperatures, for example below 4° C. or above 70° C., and these transitions are reversible. Such viscosity changes are not typically dependent on the polymer concentration, but are rather temperature-dependent. Without wishing to be bound by any theory, it is believed that the active ingredients of the present pharmaceutical formulations exhibit surfactant-like characteristics, due in part to their permanent positive charge, and thus these active ingredients interact with the poloxamers in unexpected ways. It is believed that the active ingredients of the present pharmaceutical formulations typically do not pass through the cell membrane, due to their permanent positive charge. However, it is further believed that they will penetrate into the cell, in therapeutically effective amounts, when access is afforded via open TRPV1 channels. This presents one advantage of the present disclosure as compared to their corresponding neutral molecules that are believed to freely penetrate all cell membranes.

For example, as demonstrated in Example 1 below, substantially no decrease in viscosity was observed during stability tests at 40° C./75% RH for a pharmaceutical formulation of the disclosure comprising the poloxamer Kolliphor P 407 and COMPOUND A bromide salt. As can be seen from Example 1, at 20-22% w/w of the poloxamer, the pharmaceutical formulator of the disclosure was less viscous than expected, and demonstrated a lotion-like viscosity rather than a substantially gel-like consistency. However, a formulation that was identical except for the presence of the active ingredient, using 22% poloxamer, demonstrated a gel-like consistency.

The present disclosure thus provides for stable or stabilized pharmaceutical formulations comprising an active ingredient of the disclosure as described herein, for example stable or stabilized formulations comprising one or more compounds of Formula (I), (II) or (III), or enantiomers, pharmaceutically acceptable salts thereof. The stability of a formulation according to the present disclosure can be determined, for example, by measuring the physical state of the formulation, including the viscosity and presence of any discoloration. In one embodiment, the active ingredient remains in a substantially gel-like after storage for predetermined times and under predetermined conditions.

As used herein, a formulation of the disclosure which is “substantially gel-like” means that the formulation generally has the characteristics of a gel, including exhibiting substantially no flow when in the steady-state.

As discussed above, the active ingredient of the present disclosure is maintained in substantially gel-like form by combining the active ingredients with one or more hydrophilic nonionic surfactants such as poloxamers. Suitable poloxamers for use according to the present disclosure include Pluronic®, Synperonic® or Kolliphor® P407.

Poloxamers are non-ionic poly (ethylene oxide) (POE)-poly (propylene oxide) POP copolymers. They are used in pharmaceutical formulations as surfactants, emulsifying agents, solubilizing agents, dispersing agents, and in vivo absorbance enhancers. Poloxamers are typically synthetic triblock copolymers with similar chemical structures but with different molecular weights and different relative compositions of the hydrophilic POE and hydrophobic POP components.

In some embodiments, the formulations of the disclosure are stable when subject to predetermined conditions for predetermined times. For example, pharmaceutical formulations of the disclosure can be stored at various predetermined temperatures and relative humidities for defined or predetermined time periods, for example in an open or closed container. In some embodiments, formulations of the disclosure are stable upon storage at about 5, 25, 30, 37 or 40 degrees Celsius and about 0%, 5%, 10%, 15%, 20%, 25%, 30%, 35%, 40%, 45%, 50%, 55%, 60%, 65%, 70%, 75%, 80%, 85%, 90%, 95% or 100% relative humidity for a period of at least about 0.5, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 20, 25, 30, 35, 40, 45, 48, 50, 51, 52, 53, 55 or 60 hours 1 week, 2 weeks, 3 weeks or 4 week; 1 month, 2 months, 3 months, 4 months, 5 months, 6 months, 7 months, 8 months, 9 months, 10 months, 11 months or 12 months.

In certain embodiments, formulations of the disclosure are stable upon storage in an open or closed container at: about 30 degrees Celsius and about 90 percent relative humidity for a period of at least about 20 hours; about 40 degrees Celsius and about 60 percent relative humidity for a period of at least about one week, two weeks or three weeks; about 40 degrees Celsius and about 75 percent relative humidity for a period of at least about one week, two weeks or three weeks; about 25 degrees Celsius and about 60 percent relative humidity for a period of at least about one month; about 40 degrees Celsius and about 75 percent relative humidity for a period of at least 3 months; about 25 degrees Celsius and about 75 percent relative humidity for a period of at least about 3 months; or 5 degrees Celsius at any relative humidity for a period of at least about three months. In some embodiments, “storage in an open container” means that the container was opened twice a day for a given period of time, for example up to four weeks, but was otherwise left closed.

In certain embodiments, pharmaceutical formulations of the disclosure comprise an active ingredient of the disclosure, such as (R)-2-(2-((2,3-dihydro-1H-inden-2-yl)(o-tolyl)amino)ethyl)-1,1-dimethylpiperidin-1-ium (sometimes referred to herein and in the Figures as “COMPOUND A” or “CMPD A”) or pharmaceutically acceptable salts thereof. In some embodiments, the pharmaceutically acceptable salt of COMPOUND A is the bromide salt. In some embodiments, the pharmaceutically acceptable salt of COMPOUND A is the sulfate salt. In some embodiments, the pharmaceutically acceptable salt of COMPOUND A is the methanesulfonate salt. In some embodiments, the pharmaceutical formulations can be formed into dosage forms. In such dosage forms of the disclosure, the amount of active ingredient comprising the dosage form can be any suitable amount, for example about 0.1, 0.3, 0.5, 0.7, 1, 1.5, 2, 2.5, 5, 10, 15, 20, 25, 30, 35, 40, 45, 50, 55, 60, 65, 70, 75, 80, 85, 90, 95, 96, 97, 98, 99 or 100 mg per unit dosage form. In certain embodiments, dosage forms of the disclosure comprise about 25, 50, 75, 80 or 100 mg of the active ingredient per dosage form. In some embodiments, the active ingredient in the pharmaceutical formulations of the disclosure can comprise an amount of about 0.1 to 7.5 percent by weight, for example about 0.1, 0.3, 0.5, 1, 1.5, 2, 2.5, 5, or 7.5 percent by weight. In another embodiment, the active ingredient comprises about 1 percent or about 3 percent of the pharmaceutical formulation by weight.

Although exemplary amounts or ranges for the active ingredient and poloxamer are given, pharmaceutical formulations of the disclosure can comprise any amount of these components suitable for the purposes of obtaining the desirable pharmacologic and stability properties as described herein. In addition to the stabilizing polymer and the active ingredient, pharmaceutical compositions of the disclosure can also comprise other pharmaceutically acceptable excipients, for example adjuvants, antioxidants, binders, buffers, coloring agents, diluents, emulsifiers, emollients, encapsulating materials, fillers, glidants, lubricants, metal chelators, osmo-regulators, pH adjustors, preservatives, solubilizers, sorbents, stabilizers, fragrances, surfactants, suspending agents, thickening agents, or viscosity regulators. Suitable excipients for use in pharmaceutical compositions of the disclosure are described, for example, in the “Handbook of Pharmaceutical Excipients”, 5th Edition, Eds.: Rowe, Sheskey, and Owen, APhA Publications (Washington, D.C.), Dec. 14, 2005, the disclosure of which is incorporated herein by reference.

In certain embodiments, pharmaceutical compositions of the disclosure can be formed into a gel for topical application.

The pharmaceutical formulations of the disclosure can be formulated for administration as a single application or as multiple applications for continuous or periodic discontinuous administration. For continuous administration, a kit can include the pharmaceutical formulations of the disclosure in individual unit dosage forms (e.g., in separate single-use tubes, sachets or the like), and optionally instructions for administering the individual unit dosage forms, for example, more than once daily, daily, weekly, or monthly, for a predetermined length of time or as prescribed.

Suitable packages or containers are known in the art for holding and dispensing pharmaceutical formulations for periodic topical application. In one embodiment, the package comprises indicators for each administration period. In another embodiment, the package comprises a labeled tubes, transdermal patches, or bandages for minor wounds such as Band-aid® brand adhesive bandages. The kits of the disclosure can also comprise a means for containing any type of packaging that houses the unit dosage forms, for example patches, tubes or sealed pouches, which can (for example) be held in close confinement for commercial sale such as, e.g., injection or blow-molded plastic containers into which the patches, tubes or sealed pouches are retained.

The pharmaceutical compositions, dosage forms and kits of the disclosure are useful in treating conditions which are associated with pruritus and/or pain. Though not wishing to be bound by the following theory, it is believed that indiscriminate blockage of sodium ion channels leads to inefficient pain management as a negative consequence of this route is motor impairment.

The disclosure thus provides a method of treating pruritus and/or pain by selective regulation of sodium ion channels in a subject, comprising administering to the subject a therapeutically effective amount of an active ingredient in one or more dosage forms, wherein the dosage forms comprise a pharmaceutical formulation comprising an active ingredient in substantially gel-like form and one or more stabilizing hydrophilic non-ionic surfactants, wherein the active ingredient comprises a compound of the Formula (I), (II) or (III), and wherein the pharmaceutical formulation remains in substantially gel-like form after storage of the pharmaceutical formulation for a predetermined time and under predetermined conditions.

As used herein, a therapeutically effective amount of an active ingredient of the disclosure when used for the treatment inflammation, pruritus and/or pain, and for treating conditions for which the signs and symptoms include inflammation, pruritis and/or pain is, for example, an amount which may reduce inflammation, pain, itch or other related discomfort.

As described herein, a therapeutically effective amount of an active ingredient of the disclosure when used for the treatment of inflammation, pruritus and/or pain, and for treating conditions for which the signs and symptoms include inflammation, pruritis and/or pain is an amount which may delay the onset of or reduce the severity or duration of inflammation, itch and/or pain, or which mitigates one or more symptoms of inflammation, itch, pain, dry, scaly or cracked skin, or other related discomfort. For treatment of inflammation, itch and/or pain, efficacy can be measured, for example, by a reduction in physiologic signs of discomfort (e.g., redness, swelling, heat) or by measuring changes in the levels of inflammation, pain, discomfort, irritation or scratching.

In some embodiments, a therapeutically effective amount of the pharmaceutical compositions of the disclosure can be used to treat inflammatory skin conditions such as atopic dermatitis and hand and foot eczema (including chronic hand eczema), and any of their associated signs or symptoms, such as inflammation, pain and/or pruritus. In other embodiments, a therapeutically effective amount of the pharmaceutical compositions of the disclosure can be used to treat conditions such as postherpetic itch, dermatitis herpetiformis, postherpetic neuralgia, HIV-associated distal sensory polyneuropathy, prurigo nodularis, pemphigus vulgaris, hypertrophic scar, chronic prurigo, uremic pruritus or notalgia paresthetica.

and any of their associated signs or symptoms, such as inflammation, pain and/or pruritis. In still other embodiments, a therapeutically effective amount of the pharmaceutical compositions of the disclosure can be used to treat any condition involved with or which can be ameliorated by blocking sodium channels, for example to reduce pain, pruritus and skin inflammation.

In some embodiments, a subject being treated with a therapeutically effective amount of the pharmaceutical compositions of the disclosure may experience one, some or all of the following: reduction or blocking of the inflammatory response (for example in inflammatory skin conditions; reduction or blocking of pain; emollient-like relief to damaged (including dry) skin; restoration of the skin barrier in conditions where the skin barrier is compromised; reduction in the need for topical corticosteroid use, which can result in reduced topical corticosteroid adverse events such as thinning of the skin; enhancement of keratinocyte proliferation in skin conditions where keratinocyte proliferation is inhibited; stimulation of wound repair, for example by normalizing the wound bed in acute and chronic wounds including venous leg ulcers and diabetic leg ulcers; improvement of the skin appearance.

The therapeutically effective amount of a pharmaceutical formulation of the disclosure provided to a subject will vary depending upon the purpose of the administration, the state of the patient, level of pain and/or itch, and the like. As used herein, “subject” includes any human or non-human animal in need of treatment with the pharmaceutical formulations of the disclosure. In one embodiment, a subject is any human in need of treatment with the formulations of the disclosure (sometimes referred to herein as a “patient”). In another embodiment, a subject is any species of dog. A therapeutically effective amount of the active ingredient in the pharmaceutical formulations of the disclosure can be determined by an ordinarily skilled physician, veterinarian or other medical professional, taking into account certain variables, including the specific condition and the size, age, weight, gender, disease penetration, previous treatment and response pattern of the subject.

In one embodiment, the pharmaceutical formulation is administered topically in a gel form. For example, the present formulations can be provided as a unit dose, for example as a single unit topical application, which when applied comprises a therapeutically effective amount. In one embodiment, a unit dose comprising the pharmaceutical formulation of the disclosure can be administered once daily or multiple times daily, for example, 1 to 6 times in a 12 or 24 hour period. If multiple unit doses are administered in a given time period, they can be administered at substantially even time intervals. For example, if two unit doses are administered in a 12 hour period, they can be given to the patient 6 hours apart. Multiple unit doses are administered in a given time period can also be administered at substantially uneven time intervals. In one embodiment, a unit dose comprises a dosage form of the disclosure in the form of a single application gel tube.

A suitable daily (i.e., 24 hour time period) dose according to methods of the disclosure, whether given all at once or in multiple administrations, can depend on the specific method of treatment and condition treated. In one embodiment, a suitable daily dose, whether given all at once or in multiple administrations, is between about 5 μg/cm² to about 1000 μg/cm², about 15 μg/cm² to 150 μg/cm², or about 50 μg/cm² to about 100 μg/cm².

In one embodiment, pharmaceutical formulations of the present disclosure can be applied at an application rate (i.e., total amount of active ingredient applied topically to a subject or patient) of about 80 μg/cm² to about 820 μg/cm². In one embodiment, pharmaceutical formulations of the present disclosure can be applied topically to a subject or patient at an application rate of about: 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798 or 800 μg/cm². In one embodiment, pharmaceutical formulations of the present disclosure can be applied topically to a subject or patient at an application rate of 82, 164, 328 or 492 μg/cm². In these and other embodiments, pharmaceutical formulations of the present disclosure can be applied topically on the volar forearm of a subject or patient over a 10 cm² area. In one embodiment, the subject or patient can self-administer the pharmaceutical formulation, which can result in a variability in the actual application rate of about 0.5 to 10%, for example about 1 to 8% or about 2.5 to 5%.

The following examples are given to illustrate exemplary embodiments of the present disclosure. It should be understood, however, that the present disclosure is not to be limited to the specific conditions or details described in these examples.

### EXAMPLES

While the present disclosure has been discussed in terms of certain embodiments, it should be appreciated that the present disclosure is not so limited. The embodiments are explained herein by way of example, and there are numerous modifications, variations and other embodiments that can be employed that would still be within the scope of the present disclosure.

### Example 1

### COMPOUND A Gel Formulations

Three varying prototypes of COMPOUND A bromide salt gel formulations were produced using the general method described below:
1. COMPOUND A bromide salt was first dissolved in combination of propylene glycol and water (half total % water).
2. Poloxamer (Kolliphor P 407) was added to rest of the water (chilled at 4° C.) and mixed until a clear gel was obtained.
3. COMPOUND A bromide salt solution was added to poloxamer gel. COMPOUND A solution was stirred and mixed with the poloxamer gel to produce a homogenous COMPOUND A bromide salt poloxamer gel formulation.

The tables below provide the recipes for the test and control article gels used in the Examples herein.

TABLE 1
3% COMPOUND A bromide salt -Poloxamer Gel

| Ingredients | % w/w |
|---|---|
| COMPOUND A bromide salt | 3 |
| Water | 47 |
| Propylene Glycol | 20 |
| Poloxamer (Kolliphor P 407) | 30 |
| Total | 100 |

TABLE 2
5% COMPOUND A bromide salt -Poloxamer Gel

| Ingredients | % w/w |
|---|---|
| COMPOUND A bromide salt | 5 |
| Water | 45 |
| Propylene Glycol | 20 |
| Poloxamer (Kolliphor P 407) | 30 |
| Total | 100 |

TABLE 3
7.5% COMPOUND A bromide salt -Poloxamer Gel

| Ingredients | % w/w |
|---|---|
| COMPOUND A bromide salt | 7.5 |
| Water | 42.5 |
| Propylene Glycol | 20 |
| Poloxamer (Kolliphor P 407) | 30 |
| Total | 100 |

TABLE 4
COMPOUND A bromide salt 0.3%, 1% and 3% Topical Gels

| | % (w/w) of formulation | | | | |
|---|---|---|---|---|---|
| Component | 0.3% | 1% | 3% | 7.5% | Placebo |
| COMPOUND A | 0.3^{a} | 1.0^{b} | 3.0^{c} | 7.5^{d} | — |
| Propylene glycol, USP | 20.0 | 20.0 | 20.0 | 20.0 | 20.0 |
| Poloxamer 407, NF | 30.0 | 30.0 | 30.0 | 30.0 | 30.0 |
| Methylparaben, NF | 0.05 | 0.05 | 0.05 | 0.05 | 0.05 |
| Propylparaben, NF | 0.025 | 0.025 | 0.025 | 0.025 | 0.025 |
| Sterile water for | 49.625 | 48.925 | 46.925 | 42.425% | 49.925 |
| irrigation, USP | | | | | |
| Total | 100.0 | 100.0 | 100.0 | 100.0 | 100.0 |

NF = National Formulary;
USP = United States Pharmacopiea
^{a}0.3% of COMPOUND A drug substance is equivalent to 0.25% of the active moiety (excludes bromide counterion).
^{b}1.0% of COMPOUND A drug substance is equivalent to 0.82% of the active moiety (excludes bromide counterion).
^{c}3.0% of COMPOUND A drug substance is equivalent to 2.46% of the active moiety (excludes bromide counterion).
^{d}7.5% of COMPOUND A drug substance is equivalent to 6.15% of the active moiety (excludes bromide counterion).

A 2% COMPOUND A bromide salt poloxamer gel was made by diluting the 3% COMPOUND A bromide salt gel shown in Table 4 above 1.5× with the placebo gel shown in Table 4.

The general method for making COMPOUND A bromide salt polyethylene glycol ointment (PEG Ointment) is described below:
1. COMPOUND A bromide salt was first dissolved in combination of propylene glycol and water.
2. PEG 3350 and PEG 600 were mixed and heated to 70° C.
3. COMPOUND A bromide salt solution from step (1) was added to molten PEG mixture from step (2). COMPOUND A solution was stirred and mixed with the PEG molten ointment to produce a homogenous COMPOUND A bromide salt PEG ointment of the desired percentage (w/w).

Certain problems were observed with the COMPOUND A bromide salt polyethylene glycol ointment. For example, discoloration of the ointment was observed without butyrated hydroxytoluene (BHT) in a week at 40° C./75% RH. After adding BHT 0.1% the discoloration slowed and slight yellowing was observed in 1 month at 40° C./75% RH; the ointment also melted at 40° C./75% RH when kept at stability but solidified when kept at room temperature for 15 min; and finally, the pharmacodynamic data was not convincing as compared with the poloxamer gel formulation. Table 5 below provides % w/w for COMPOUND A bromide salt polyethylene glycol ointment.

TABLE 5
Polyethylene glycol Ointment (PEG Ointment)

| Ingredients | % w/w |
|---|---|
| COMPOUND A bromide salt | 3 |
| Water | 14.9 |
| Propylene Glycol | 20 |
| PEG 3350 | 37.2 |
| PEG 600 | 24.8 |
| Butylated hydroxyl Toluene | 0.1 |
| Total | 100 |

The general method for making COMPOUND A bromide salt hydroxyethyl cellulose-based gel (HEC Gel) is described below:
1. Benzyl Alcohol was added to the desired amount of water and mixed using paddle blade for 10 minutes or until clear solution is observed.
2. COMPOUND A bromide salt was then added to the solution from step (1) until the entire API was dissolved, up to the desired percentage (w/w).
3. Hydroxy ethyl cellulose (HHX Grade) was then added and mixed until a clear gel was formed.

Certain problems were also observed with the COMPOUND A bromide salt HEC gel formulation. For Example, discoloration of the gel formulation with time in stability tests at 40° C./75% RH and a decrease in viscosity of the gel formulation at stability tests at 40° C./75% RH was observed.

In addition, other problems, including poor formulation consistency, have been found with using poloxamers at a lower concentration. In an effort to solve the discoloration and change in viscosity issues over time during stability tests, a thermo-reversible polymer was utilized. The viscosity of this polymer decreases only in extremes of temperatures such as below 4° C. or above 70° C., and these transitions are reversible. Moreover, in COMPOUND A bromide salt formulations comprising such poloxamers, substantially no decrease in viscosity was observed over stability tests at 40° C./75% RH. Surprisingly and unexpectedly, a higher percentage of the poloxamer (Kolliphor P 407) was found to solve the problems of poor consistency, and was thus used to formulate COMPOUND A bromide salt. For example, at a lower percentage of poloxamer (e.g., 20-22%), the COMPOUND A bromide salt formulation was less viscous and had a lotion-like viscosity. Without wishing to be bound by any theory, this could be due to the surfactant nature of the active ingredient, which potentially causes the thinning of the gel, as a placebo (i.e., no COMPOUND A bromide salt) with 22% poloxamer was a gel consistency. The present disclosure therefore provides an unexpected discovery that a higher percentage of poloxamer solved the problems of formulation consistency. COMPOUND A bromide salt-poloxamer gel formulations also showed little to no discoloration at lower concentrations of the COMPOUND A bromide salt. Without wishing to be bound by any theory, it is expected that poloxamer gel formulations of the disclosure comprising COMPOUND A methanesulfonate salt will exhibit little to no discoloration even at higher concentrations of the COMPOUND A methanesulfonate salt.

Poloxamer gels comprising the methanesulfonate salt of COMPOUND A are made in the same manner as described above for the COMPOUND A bromide salt, according to the following recipes:

TABLE 1
3% COMPOUND A methanesulfonate salt -Poloxamer Gel

| Ingredients | % w/w |
|---|---|
| COMPOUND A methanesulfonate salt | 3 |
| Water | 47 |
| Propylene Glycol | 20 |
| Poloxamer (Kolliphor P 407) | 30 |
| Total | 100 |

TABLE 2
5% COMPOUND A methanesulfonate salt -Poloxamer Gel

| Ingredients | % w/w |
|---|---|
| COMPOUND A methanesulfonate salt | 5 |
| Water | 45 |
| Propylene Glycol | 20 |
| Poloxamer (Kolliphor P 407) | 30 |
| Total | 100 |

TABLE 3
7.5% COMPOUND A methanesulfonate salt -Poloxamer Gel

| Ingredients | % w/w |
|---|---|
| COMPOUND A methanesulfonate salt | 7.5 |
| Water | 42.5 |
| Propylene Glycol | 20 |
| Poloxamer (Kolliphor P 407) | 30 |
| Total | 100 |

TABLE 4
COMPOUND A methanesulfonate salt
0.3%, 1% and 3% Topical Gels

| | % (w/w) of formulation | | | |
|---|---|---|---|---|
| Component | 0.3% | 1% | 3% | 7.5% |
| COMPOUND A | 0.3 | 1.0 | 3.0 | 7.5 |
| methanesulfonate salt | | | | |
| Propylene glycol, USP | 20.0 | 20.0 | 20.0 | 20.0 |
| Poloxamer 407, NF | 30.0 | 30.0 | 30.0 | 30.0 |
| Methylparaben, NF | 0.05 | 0.05 | 0.05 | 0.05 |
| Propylparaben, NF | 0.025 | 0.025 | 0.025 | 0.025 |
| Sterile water for irrigation, USP | 49.625 | 48.925 | 46.925 | 42.425% |
| Total | 100.0 | 100.0 | 100.0 | 100.0 |

NF = National Formulary;
USP = United States Pharmacopiea

All references to “COMPOUND A” in Examples 2 through 7 below indicate the bromide salt of COMPOUND A.

### Example 2

### Effects of COMPOUND A in Different Formulations

The aim of the present study was to observe and compare the effects of COMPOUND A in a HEC gel formulation with COMPOUND A in a poloxamer gel formulation and COMPOUND A in an ointment-based formulation.

In previous studies, it was determined that 3% COMPOUND A applied to the skin six hours prior to chloroquine injection in a mouse model was the optimal concentration and time for a significant reduction of scratching behavior when compared to vehicle alone. 3% COMPOUND A in each of the three vehicle formulations prepared as described in Example 1 above—poloxamer (from Table 4 above); hydroxyethylcellulose (HEC) and polyethylene glycol (Ointment) (50 μl) was applied topically to the skin 6 hours prior to the intradermal injection of chloroquine (CQ) (4 mg/ml, 50 μl). Mice in these groups did not display scratching at the site of test article or vehicle application prior to injection of CQ. Motor function (measured by placing and stepping) and pinna and corneal reflexes were normal following the application of the drug. No weakness was observed in any of the animals. Following intradermal CQ injection in the ipsilateral neck area, the number of scratches was counted and cumulative counts were recorded by an automated machine for 40 minutes.

### Husbandry

Upon receipt at the testing facility's AAALAC approved Clinical Teaching Facility Vivarium, animals were randomly separated into cages containing approved bedding material to acclimate. Room temperature was maintained within the range 65 to 82° F. and relative humidity within the range 30 to 75%. The room was illuminated with fluorescent lighting on a daily 12-hour light/dark cycle. Animals were checked a minimum of once a day. Cages were changed and cleaned a minimum of once weekly. All animals had free access to dry rodent food. Municipal tap water was freely available and water analyses reports are on file with the test facility.

Animals:

Species: Mouse

Strain: C57/B16

Source: Harlan Laboratories, Indianapolis Ind.

Number required: 48 Males

Identification: Unique Cage Identifier, Tail Identifier

Approximate weight on first day of dosing: 20-30 grams

Type of accommodation: Gang housed

Minimum period of acclimation: 2 days

Test Facility: UCSD Clinical Teaching Facility Vivarium

### Study Design

Protocols: Unless otherwise stated, the proposed study was conducted in accordance with the then-current laboratory protocols, SOPs and analytical methods of the test facility. Day 0 (zero) is designated as the first day animals receive Test/Control Article.

Randomization: Animals were randomly assigned to dosing groups based upon date of receipt at the test facility.

Dosing Groups: Table 6, below, outlines the study groups tested with Sponsor supplied Test Articles and Control.

TABLE 6
Dosing and Testing Paradigm

| | | | | Behavioral |
|---|---|---|---|---|
| | | | Intradermal | Analysis (After |
| | | Dosing | Chloroquine | 50 μL CQ ID* |
| | Number of | (Topical | (CQ) (4 mg/ | at 6 hours for |
| Test/Control Article | Animals | Application) | mL, 50 μL) | 40 minutes) |
| HEC Gel | 8 | 50 μL at Time 0 | 6 hours | Scratching |
| | | | | Counts 6-6:40 hrs |
| Poloxamer Gel | 8 | 50 μL at Time 0 | 6 hours | Scratching |
| | | | | Counts 6-6:40 hrs |
| Ointment | 8 | 50 μL at Time 0 | 6 hours | Scratching |
| | | | | Counts 6-6:40 hrs |
| HEC Gel + | 8 | 50 μL at Time 0 | 6 hours | Scratching |
| Compound A (3%) | | | | Counts 6-6:40 hrs |
| Poloxamer Gel + | 8 | 50 μL at Time 0 | 6 hours | Scratching |
| Compound A (3%) | | | | Counts 6-6:40 hrs |
| Ointment + | 8 | 50 μL at Time 0 | 6 hours | Scratching |
| Compound A (3%) | | | | Counts 6-6:40 hrs |

HEC = hydroxyethylcellulose
*ID = intradermal

Pruritus Paradigm: At time 0 mice were lightly anesthetized with isoflurane for topical application of Control or Test Article to the right lateral shoulder area. The shoulder area was shaved and a circle was marked at the site with a stencil (diameter of 15 mm). Control or Test Article was applied to the circled area. Animals remained lightly anesthetized for 10 minutes following application to allow for absorption. Approximately one hour prior to Chloroquine injection, mice were fitted with a metal band on an ipsilateral hind paw and allowed to acclimate to the band and test chamber. At 6 hours post Test/Control Article treatment, animals received an intradermal injection of 50 μL Chloroquine (4 mg/mL) in the middle of the marked circle and their scratching behavior was counted for 40 minutes. Upon completion of the scratching assessment, mice were deeply anesthetized for cardiac puncture and blood collection, and then euthanized.

Recovery: The condition of the site of injection was examined and observations of redness, swelling or abrasions due to scratching were made prior to and following scratch counting. Animals were euthanized upon completion of the final observations.

There were no unscheduled deaths in this study. Body weights were taken prior to test article administration and experimental procedures. On Day 0 animals were randomly selected for study. Prior to initiation of pruritus, animals were acclimated to the testing environment. Plasma samples were collected upon completion of the testing and the animals were sacrificed. No general behavioral assessments were made.

Pruritus Model: Animals were briefly anesthetized with isoflurane anesthesia, the right lateral scapular area was shaved and 50 μL of Control or Test Article was applied topically at the shoulder area ipsilateral to the banded paw. One hour before the predetermined pretreatment time of 6 hours mice were fitted with a thin metal band on the hind paw, and allowed to acclimate to the testing chamber (cylinder) and presence of the band prior to injection of the pruritogen (chloroquin). Animals could move freely about in the chamber. Following injection of the pruritogen, automated counting of the scratching behavior at the injection site was done using the paw motion detector (PMD) for a 40 minute interval.

Tissue Harvest: Following the completion of the pruritis study, animals in each of the drug treated groups were deeply anesthetized for collection of blood 7.5 hours after application and subsequent harvest of plasma for analysis of plasma drug concentrations.

Sample Storage: Plasma samples will be stored at −70° C. and shipped for analysis.

Statistical analysis: The data for each variable was put in tabular form (i.e. GraphPad Prism). Summary statistics (e.g., One Way Analysis of Variance (ANOVA), with Bonferroni post hoc) were computed and include group means, standard deviations and numbers of animals per group using Prism Graph Pad.

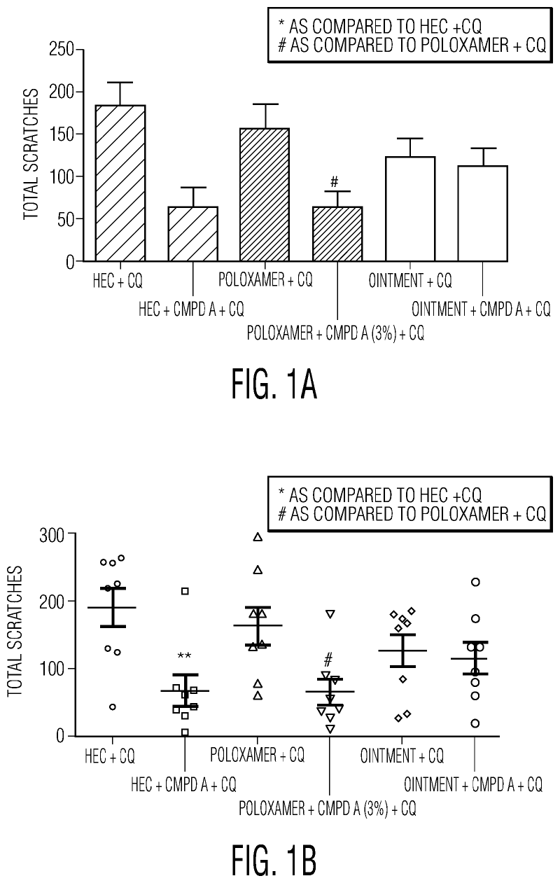

Results: Effects of COMPOUND A in different formulations (HEC, Poloxamer and Ointment) Topical application of COMPOUND A (3%) in HEC gel, 6 hours prior to intradermal injection of CQ (4 mg/ml, 50 ul) showed a significant reduction in CQ induced scratching behavior as compared to the HEC gel treated group. Similarly, COMPOUND A (3%) in Poloxamer also showed a significant decrease in CQ induced total scratches over 40 min period as compared the Poloxamer Gel treated group. However, COMPOUND A (3%) in ointment was not significantly different from the group that was treated with ointment alone. Mice in these groups did not display scratching at the site of test article or vehicle application prior to injection of CQ. Motor function (measured by placing and stepping) and pinna and corneal reflexes were normal following the application of the drug. No weakness was observed in any of the animals. FIG. 1 presents the scratching response over 40 min period time course after intradermal chloroquine in mice treated with different drug formulations.

As shown in FIG. 1, animals pretreated with vehicle showed a significant increase in the number of scratches following intradermal chloroquine. The animals pre-treated with HEC gel+COMPOUND A (3%) had significantly reduced chloroquine induced scratches as compared to animals that were pretreated only with the HEC Gel. The animals pre-treated with poloxamer gel+COMPOUND A (3%) had significantly reduced chloroquine induced scratches as compared to animals that were pretreated only with the poloxamer Gel. No differences were observed between the Ointment group and Ointment+COMPOUND A (3%) formulations on chloroquine induced scratching.

According to the results of the present experiment, poloxamer gel+COMPOUND A (3%) provides the most therapeutic potential for intervention in pruritis compared to the other agents that were tested.

### Example 3

### Effect of Various COMPOUND A Concentrations in the Present Formulations Vs. in Dimethylsulfoxide Vehicle

Different test articles comprising varying concentrations of COMPOUND A in hydroxyethyl cellulose (HEC) formulations of the specification (“Gel”) and in dimethylsulfoxide (DMSO) vehicle were tested on mice according to the pruritis model described in Example 2. The same strain of mice was used and the animals were housed in the same manner. Each treatment group consisted of 8 mice, and test articles Gel+COMPOUND A 0.3%, 1%, 3% and 5%, and DMSO+COMPOUND A 3% and 5%, were applied topically to the skin six hours prior to the intradermal injection of chloroquine (CQ) (4 mg/ml, 50 μl). The test articles used were prepared as described above in Example 1. Additional groups of 8 mice each were also pre-treated with Gel vehicle alone or DMSO alone, and another group of 8 mice did not receive vehicle, drug or CQ injection (Naïve). The site of drug application was observed approximately 24 hours following application of test and control articles for any tissue reaction. Following intradermal CQ injection in the ipsilateral neck area, the number of scratches were counted and cumulative counts were recorded by the automated machine for 40 minutes. Following observation, animals were euthanized. In selected groups (Gel+3% COMPOUND A and Gel+5% COMPOUND A) plasma was harvested at 7 hours following the scratch counting. No unscheduled deaths were observed. Harvested plasma samples were stored at −20° C. prior to analysis. Data was collected, tabulated and analyzed as described in Example 2. The experimental design is shown in Table 7 below.

TABLE 7
Experimental Design

| | | | | Behavioral |
|---|---|---|---|---|
| | | | Intradermal | Analysis (After |
| | | Dosing | Chloroquine | 50 μl CQ ID* |
| | Mouse | (Topical | (CQ) (4 mg/ | at 6 hours for |
| Test/Control Article | Number | Application) | ml, 50 μl) | 40 minutes) |
| Naive | 8 | n/a | n/a | Scratching |
| | | | | Counts 6-6:40 hrs |
| Naive | 8 | n/a | 6 hours | Scratching |
| | | | | Counts 6-6:40 hrs |
| DMSO | 8 | 50 μl at Time 0 | n/a | Scratching |
| | | | | Counts 6-6:40 hrs |
| DMSO | 8 | 50 μl at Time 0 | 6 hours | Scratching |
| | | | | Counts 6-6:40 hrs |
| DMSO + Compound A | 8 | 50 μl at Time 0 | n/a | Scratching |
| (3%) | | | | Counts 6-6:40 hrs |
| DMSO + Compound A | 8 | 50 μl at Time 0 | n/a | Scratching |
| (5%) | | | | Counts 6-6:40 hrs |
| DMSO + Compound A | 8 | 50 μl at Time 0 | 6 hours | Scratching |
| (3%) | | | | Counts 6-6:40 hrs |
| DMSO + Compound A | 8 | 50 μl atTime 0 | 6 hours | Scratching |
| (5%) | | | | Counts 6-6:40 hrs |
| Gel | 8 | 50 μl at Time 0 | n/a | Scratching |
| | | | | Counts 6-6:40 hrs |
| Gel | 8 | 50 μl at Time 0 | 6 hours | Scratching |
| | | | | Counts 6-6:40 hrs |
| Gel + Compound A (0.3%) | 8 | 50 μl at Time 0 | 6 hours | Scratching |
| | | | | Counts 6-6:40 hrs |
| Gel + Compound A (1%) | 8 | 50 μl at Time 0 | 6 hours | Scratching |
| | | | | Counts 6-6:40 hrs |
| Gel + Compound A (3%) | 8 | 50 μl at Time 0 | n/a | Scratching |
| | | | | Counts 6-6:40 hrs |
| Gel + Compound A (5%) | 8 | 50 μl at Time 0 | n/a | Scratching |
| | | | | Counts 6-6:40 hrs |
| Gel + Compound A (3%) | 8 | 50 μl at Time 0 | 6 hours | Scratching |
| | | | | Counts 6-6:40 hrs |
| Gel + Compound A (5%) | 8 | 50 μl at Time 0 | 6 hours | Scratching |
| | | | | Counts 6-6:40 hrs |

*ID = intradermal

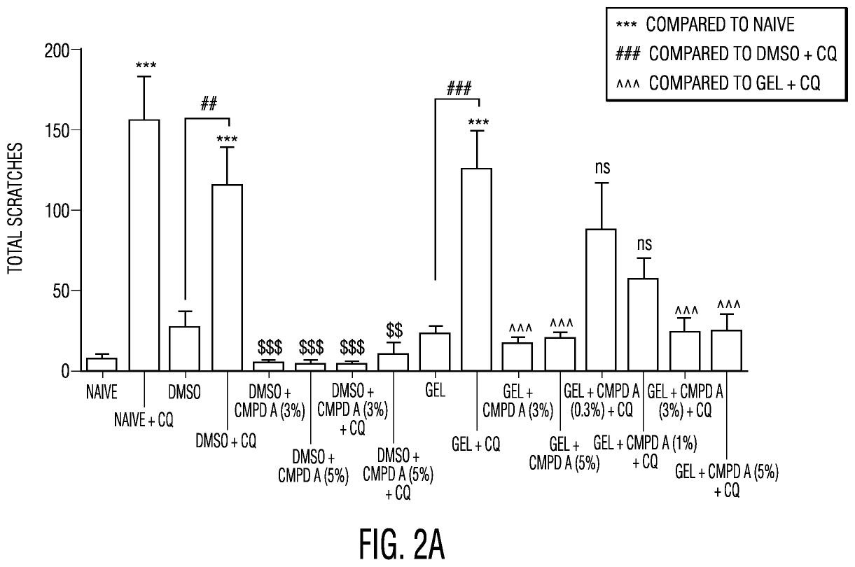

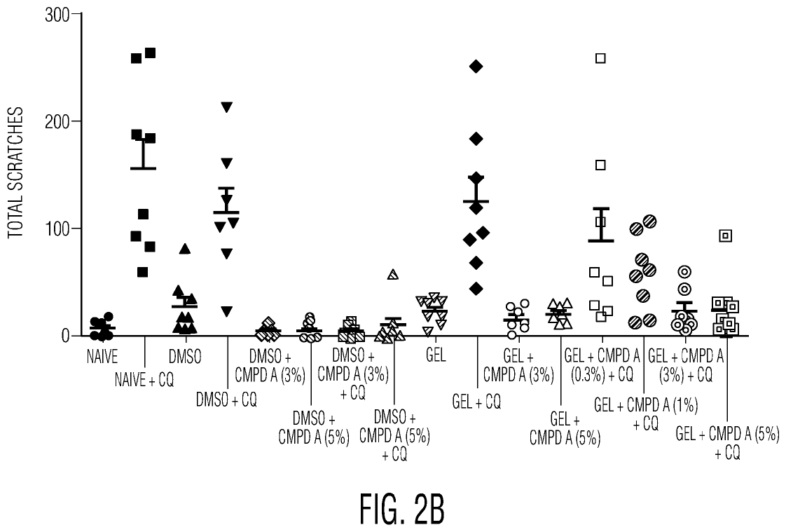

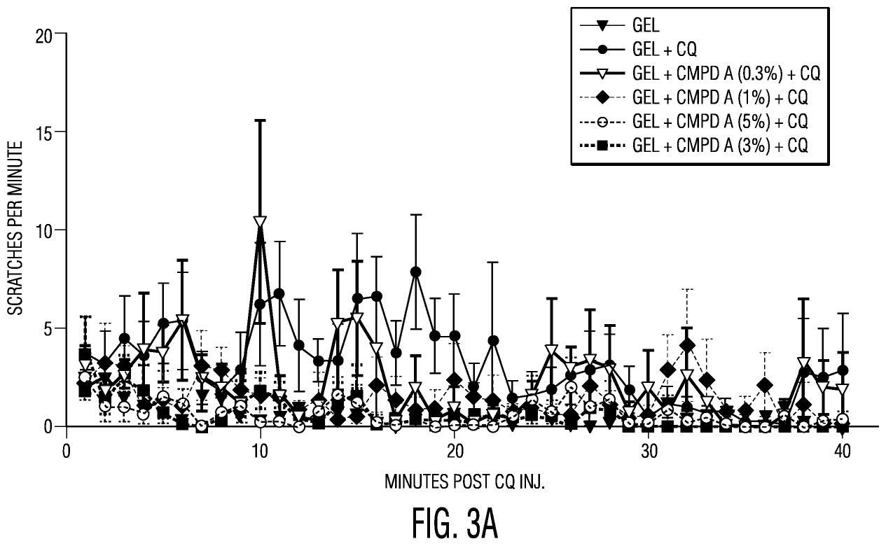

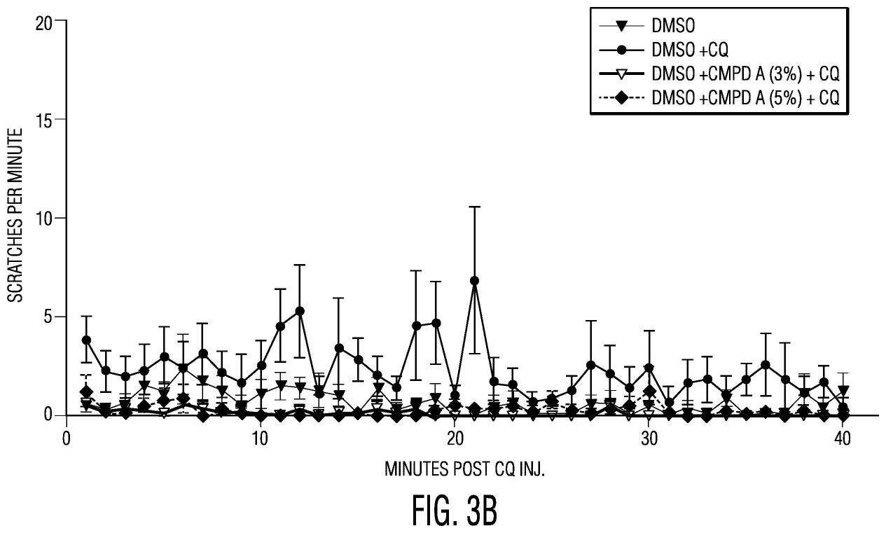

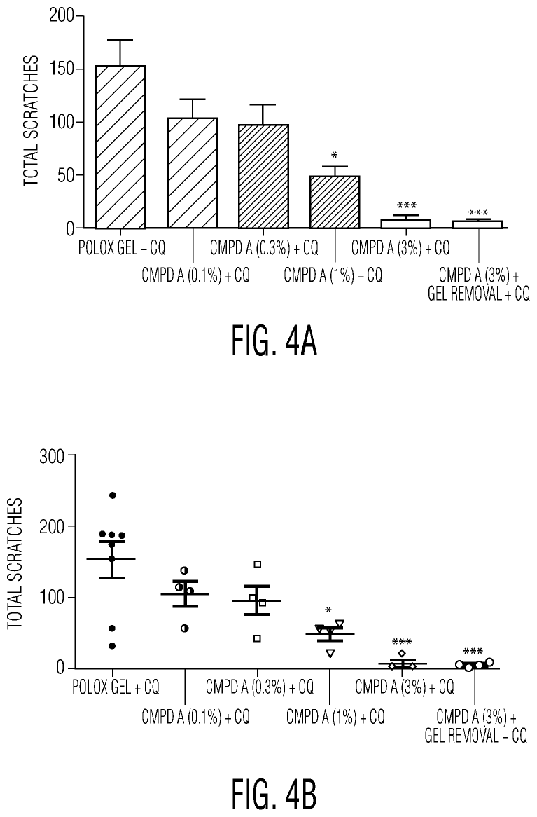

Some mice throughout all groups displayed mild scratching at the site of test article or vehicle application beginning 30 minutes after the application of the test article and lasting for approximately 2 hours. One animal in the Gel+COMPOUND A 3% group was removed due to excessive scratching and skin lesions seen at the 6 hour time point. Motor function (measured by placing and stepping) and pinna and corneal reflexes were normal following the application of the drug. Some animals in the Gel+COMPOUND A 3% and 5% groups showed very mild weakness when handled; however, this weakness did not affect the motor grip strength test and there was no difference in grip strength between the groups in the treated or non-treated groups. As can be seen in FIG. 2, naïve animals or animals pretreated with Gel or DMSO vehicles showed a significant increase in the number of scratches following intradermal chloroquine injections as compared to the groups that did not receive chloroquine (***P<0.001). The animals pre-treated with DMSO+COMPOUND A 3% and 5% showed a complete inhibition of chloroquine induced scratches and this reduction was statistically significant as compared to the group that were pretreated only with the vehicle ($$ P<0.01, $$$ P<0.001). The group of animals pre-treated with Gel+COMPOUND A 0.3%, 1%, 3% and 5% showed a dose dependent effect on chloroquine induced scratching. Treatment with Gel+COMPOUND A 0.3% and 1% did not significantly inhibit CQ induced scratching (ns), whereas treatment with Gel+COMPOUND A 3% and 5% showed a significant attenuation of CQ induce scratches (\hat{ }\hat{ }\hat{ }P<0.001). The test article alone (DMSO+COMPOUND A and Gel+COMPOUND A) did not affect the area of the skin where the test article was applied. FIGS. 3A and 4A show scratches/min following CQ treatment for the test article and DMSO treatment groups.

### Example 4

### Tachyphylaxis Studies on the Antipruritic Action of COMPOUND A Formulations

Dosing Groups and Experimental Phases. This tachyphylaxis study was performed in three phases. The first phase assessed the effect of successive dosing of COMPOUND A at a higher concentration (3%, 1% and placebo) on CQ induced scratching behavior. Phase 2 investigated the effect of successive dosing of COMPOUND A at a lower concentration (0.3%, 0.1% and placebo) on CQ induced scratching behavior. Phase 3 confirmed the findings of Phase 1 and Phase 2. Test and control articles were made with poloxamer gel and prepared as shown above in Example 1, Table 4.

Phase 1, Tachyphylaxis. Phase 1 examined the effect of the successive dosing of COMPOUND A (3%, 1% and placebo) and a COMPOUND A (3%) gel removal group for five days with scratching behavior tested on day 5 after treatment. The test articles were prepared as described in Example 1 above. Animals were pretreated with test or control article for 5 consecutive days. Three hours after treatment on day 5, animals were injected with 50 μL Chloroquine (CQ; 4 mg/mL) at the site of test or control article application, and scratching behavior was recorded for 40 minutes. In the gel removal group, the test article was removed 3 hours after application for consecutive 5 days. Table 8 outlines the study groups that were tested in Phase 1.

TABLE 8
Tachyphylaxis Study Design, Phase 1

| | | | | Behavioral |
|---|---|---|---|---|
| | | Dosing | Intradermal | Analysis (After |
| Study | Test/Control Article | (Topical | Chloroquine (CQ) | 50 μl CQ ID* |
| Day | (n = 4 each) | Application) | (4 mg/ml, 50 μl) | for 40 min) |
| 1 | Poloxamer Gel | 50 μl at Time 0 | N/A | N/A |
| | Gel + Compound A | 50 μl at Time 0 | N/A | N/A |
| | (3%), Gel removal | | | |
| | after 3 hrs | | | |
| | Gel + Compound A | 50 μl at Time 0 | N/A | N/A |
| | (1%) | | | |

Phase 2, Tachyphylaxis. Phase 2 examined the effect of the successive dosing of COMPOUND A (0.1%, 0.3% and placebo) on scratching behavior on day 5 following the treatment. The test articles were prepared as described in Example 1 above. Animals were pretreated with test or control article for 5 consecutive days. Three hours after treatment on day 5, animals were injected with 50 μL Chloroquine (4 mg/mL) at the site of test or control article application and scratching behavior recorded for 40 minutes. Table 9, below, outlines the study groups that were tested in Phase 2.

TABLE 9
Tachyphylaxis Study Design, Phase 2

| | | | | Behavioral |
|---|---|---|---|---|
| | | Dosing | Intradermal | Analysis (After |
| Study | Test/Control Article | (Topical | Chloroquine (CQ) | 50 μl CQ ID* |
| Day | (n = 4 each) | Application) | (4 mg/ml, 50 μl) | for 40 min) |
| 1 | Poloxamer Gel | 50 μl at Time 0 | N/A | N/A |
| | Gel + Compound A | 50 μl at Time 0 | N/A | N/A |
| | (0.1%) | | | |
| | Gel + Compound A | 50 μl at Time 0 | N/A | N/A |
| | (0.3%) | | | |
| 2 | Poloxamer Gel | 50 μl at Time 24 | N/A | N/A |
| | | hr | | |
| | Gel + Compound A | 50 μl at Time 24 | N/A | N/A |
| | (0.1%) | | | |
| | Gel + Compound A | 50 μl at Time 24 | N/A | N/A |
| | (0.3%) | | | |
| 3 | Poloxamer Gel | 50 μl at Time 48 | N/A | N/A |
| | | hr | | |
| | Gel + Compound A | 50 μl at Time 48 | N/A | N/A |
| | (0.1%) | | | |
| | Gel + Compound A | 50 μl at Time 48 | N/A | N/A |
| | (0.3%) | | | |
| 4 | Poloxamer Gel | 50 μl at Time 72 | N/A | N/A |
| | | hr | | |
| | Gel + Compound A | 50 μl at Time 72 | N/A | N/A |
| | (0.1%) | | | |
| | Gel + Compound A | 50 μl at Time 72 | N/A | N/A |
| | (0.3%) | | | |
| 5 | Poloxamer Gel | 50 μl at Time 96 | 3 hours | Scratching Counts |
| | | hr | | 3-3:40 hrs |
| | Gel + Compound A | 50 μl at Time 96 | 3 hours | Scratching Counts |
| | (0.1%) | | | 3-3:40 hrs |
| | Gel + Compound A | 50 μl at Time 96 | 3 hours | Scratching Counts |
| | (0.3%) | | | 3-3:40 hrs |

Phase 3, Tachyphylaxis. Phase 3 examined the effect of the successive dosing of COMPOUND A (0.1%, 0.3%, 1%, 3%, placebo and placebo group without CQ) on CQ induced scratching behavior on day 5 following the treatment. Animals were pretreated with test or control article for 5 consecutive days. Three hours after treatment on day 5, animals were injected with 50 μL Chloroquine (CQ; 4 mg/mL) at the site of test or control article application and scratching behavior recorded for 40 minutes. Table 10 below, outlines the study groups to be tested in Phase 3.

TABLE 10
Tachyphylaxis Study Design, Phase 3

| | | | | Behavioral |
|---|---|---|---|---|
| | | Dosing | Intradermal | Analysis (After |
| Study | Test/Control Article | (Topical | Chloroquine (CQ) | 50 μl CQ ID* |
| Day | (n = 8 each) | Application) | (4 mg/ml, 50 μl) | for 40 min) |
| 1 | Poloxamer Gel | 50 μl at Time 0 | N/A | N/A |
| | Poloxamer Gel | 50 μl at Time 0 | N/A | N/A |
| | Gel + Compound A | 50 μl at Time 0 | N/A | N/A |
| | (0.1%) | | | |
| | Gel + Compound A | 50 μl at Time 0 | N/A | N/A |
| | (0.3%) | | | |
| | Gel + Compound A | 50 μl at Time 0 | N/A | N/A |
| | (1%) | | | |
| | Gel + Compound A | 50 μl at Time 0 | N/A | N/A |
| | (3%) | | | |
| | Poloxamer Gel | 50 μl at Time 24 | N/A | N/A |
| 2 | Poloxamer Gel | 50 μl at Time 24 | N/A | N/A |
| | Gel + Compound A | 50 μl at Time 24 | N/A | N/A |
| | (0.1%) | | | |
| | Gel + Compound A | 50 μl at Time 24 | N/A | N/A |
| | (0.3%) | | | |
| | Gel + Compound A | 50 μl at Time 24 | N/A | N/A |
| | (1%) | | | |
| | Gel + Compound A | 50 μl at Time 24 | N/A | N/A |
| | (3%) | | | |
| 3 | Poloxamer Gel | 50 μl at Time 48 | N/A | N/A |
| | Poloxamer Gel | 50 μl at Time 48 | N/A | N/A |
| | Gel + Compound A | 50 μl at Time 48 | N/A | N/A |
| | (0.1%) | | | |
| | Gel + Compound A | 50 μl at Time 48 | N/A | N/A |
| | (0.3%) | | | |
| | Gel + Compound A | 50 μl at Time 48 | N/A | N/A |
| | (1%) | | | |
| | Gel + Compound A | 50 μl at Time 48 | N/A | N/A |
| | (3%) | | | |
| | Poloxamer Gel | 50 μl at Time 72 | N/A | N/A |
| | Poloxamer Gel | 50 μl at Time 72 | N/A | N/A |
| 4 | Gel + Compound A | 50 μl at Time 72 | N/A | N/A |
| | (0.1%) | | | |
| | Gel + Compound A | 50 μl at Time 72 | N/A | N/A |
| | (0.3%) | | | |
| | Gel + Compound A | 50 μl at Time 72 | N/A | N/A |
| | (1%) | | | |
| | Gel + Compound A | 50 μl at Time 72 | N/A | N/A |
| | (3%) | | | |
| 5 | Poloxamer Gel | 50 μl at Time 96 | N/A | Scratching Counts |
| | | | | 3-3:40 hrs |
| | Poloxamer Gel | 50 μl at Time 96 | 3 hours | Scratching Counts |
| | | | | 3-3:40 hrs |
| | Gel + Compound A | 50 μl at Time 96 | 3 hours | Scratching Counts |
| | (0.1%) | | | 3-3:40 hrs |
| | Gel + Compound A | 50 μl at Time 96 | 3 hours | Scratching Counts |
| | (0.3%) | | | 3-3:40 hrs |
| | Gel + Compound A | 50 μl at Time 96 | 3 hours | Scratching Counts |
| | (1%) | | | 3-3:40 hrs |
| | Gel + Compound A | 50 μl at Time 96 | 3 hours | Scratching Counts |
| | (3%) | | | 3-3:40 hrs |

For all three phases, the mice were subjected to the pruritis model as described in Example 2. Upon completion of the scratching assessment mice were placed in their home cage and examined at 24 hours for any adverse skin reactions. Phase 3 animals were euthanized for blood collection following completion of the scratching counts. The condition of the site of injection was examined and observations of redness, swelling or abrasions due to scratching were made following scratch counting and at 24 hours post drug application Animals were euthanized upon completion of the final observations. One mouse died during anesthesia on day 5 after application of 3% drug and one mouse was euthanized because of weight loss. Blood plasma was collected on day 5 following the behavior in the Phase 3 animals. Plasma samples were stored on dry ice until analysis. Data was collected, tabulated and analyzed as described in Example 1, and the results of each phase are discussed below.

This study was performed in three phases, to examine the effect of the successive dosing of poloxamer gel formulations according to the present specification, COMPOUND A (1%) COMPOUND A (3%) and COMPOUND A (3% with gel removal 3 hours after application) on scratching behavior after 5 days of treatment. During Phase 1 of this study, four groups of mice (each n=4) received topical poloxamer gel, COMPOUND A (1%) and COMPOUND A (3%) drug. One group received topical application of COMPOUND A (3%), which was removed after 3 hours. To remove the formulation, a piece of gauze that had been saturated with distilled water was used to gently wipe (not rub) the application site from the rostral end to the caudal end 3 times. The skin was then patted dry with clean gauze. If there were lesions present on the skin, then extra care is taken to avoid irritation or redness.

This treatment was continued for 5 days without any CQ injections. On day 5 all these mice received intradermal CQ 3 hours after the last application of the drug (n=4). There was a slight drop in the weight of COMPOUND A treated mice; however, this was not drastic as compared to the first study. (Note that there were no CQ injections until day 5.) By day 3 the COMPOUND A treated mice had a scaly texture at the site of application. On Day 4 and 5 slight redness was observed in the COMPOUND A 3% gel treated mice (no gel removal at 3 hours). No signs of lethargy or distress were observed. Following CQ injections on day 5 these mice were observed until day 9 and they all recovered their weight and from their redness.

Tachyphylaxis: Phase 2. Three groups of mice (n=4 each) received poloxamer gel with COMPOUND A (0.1%) and COMPOUND A (0.3%). This treatment was continued for 5 days without any CQ injections and their weight was monitored. On day 5 all these mice received intradermal CQ 3 hours after the last application of the drug. No statistical significance was observed, probably because of the small experimental group size (n=4). No weight loss was observed in any of COMPOUND A treated mice. On day 3, one mouse in the COMPOUND A 0.1% treatment group developed a slight chaffing at the application site. On day 4 another mouse from the COMPOUND A 0.1% treatment group had a scaly/scab spot at the site of application, which was observed on day 5 as well. This scaly texture then faded following day 7. FIGS. 4A and 4B, respectively, show pooled data from Phases 1 and 2 for the total number of scratches following CQ injection for a 40 minute period on day 5.

Phase 3. This phase of the study examined the effect of the successive dosing of COMPOUND A (0.1%, 0.3%, 1%, 3%), placebo and placebo without CQ, on scratching behavior on day 5 following the treatment. Their weight was monitored. Following the successive dosing, the COMPOUND A 0.3%, 1% and 3% treatment groups showed a significant reduction in the number of scratches following intradermal CQ. The 3% group, however, showed signs of distress, dehydration and loss of weight starting from day 3 and exhibited 20% weight loss at Day 4. Due to this, a few animals were not applied with 3% gel on day 4 and were administered with subcutaneous fluids. On day 5 two mice from this group were sacrificed prior to the behavior study, as more than a 20% weight loss was observed. Another observation that was noted following topical application of the drug was that, at the local site of application following application of the COMPOUND A 3% formulation, the skin was scaly, reddish and by day 5 the skin developed a scab like texture. The COMPOUND A 1% gel also produced these effects on the skin; however, to a lesser extent than the 3% gel group. An additional group of 4 mice was added, in which the mice were shaved 72 hours prior to administering the 3% gel (indicated in the figures with a #). This group of mice also showed weight loss and reddish color and scabby texture of the skin.

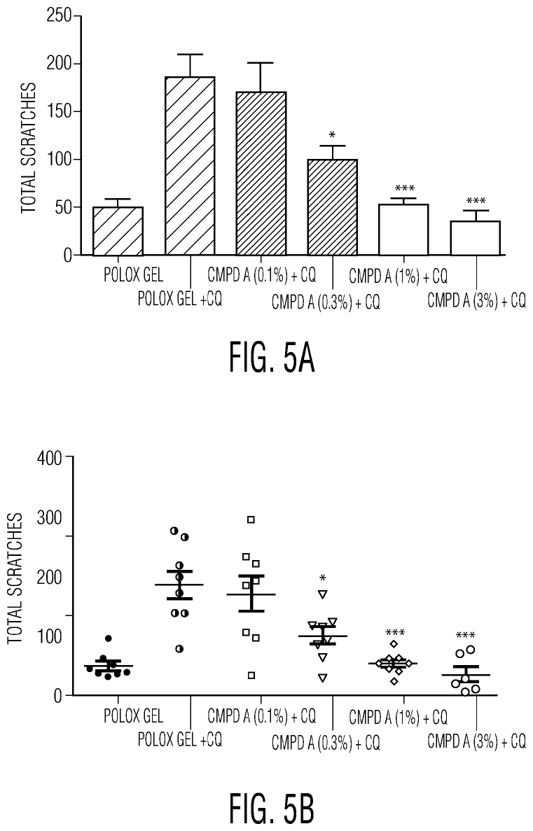

Phase 3 was undertaken as a confirmation study for the results obtained from Phase 1. In this replication, some inconsistencies were observed, as the 3% group in this Phase 3 showed a significant weight loss and signs of distress and dehydration following the drug application starting from day 3. Although weight loss was observed in Phase 1 with the 3% group, it was not as prominent or significant as that which was observed in Phase 3. Similarly, application of gel with 1% COMPOUND A resulted in some redness and scaling of the skin in Phase 3 which was not observed in the 1% COMPOUND A group in Phase 1. There was no change in the protocol or the experimental set up between the two phases, except that new batch of test articles was obtained to conduct Phase 3. FIGS. 5A and 5B, respectively, show total number of scratches following CQ injection over a 40 min period on day 5.

### Example 5

### Assessment of the Dose and Time Course of the Activity of Antipruritogen Formulations of COMPOUND A

This experiment was conducted to demonstrate the time course and effect of formulations of the present specification with 1%, 2% and 3% COMPOUND A in hydroxyethyl cellulose gel prepared as shown in Example 1 above (“Gel+COMPOUND A 1%,” “Gel+COMPOUND A 2%” and “Gel+COMPOUND A 3%”). The carrier gel alone, with no drug, is referred to as “Gel.” Gel and Gel+COMPOUND A 1%,” “Gel+COMPOUND A 2%” and “Gel+COMPOUND A 3% were applied topically to the skin of groups of 8 mice, each of which were subjected to chloroquine injection (CQ; 4 mg/ml, 50μl) according to the pruritis model described in Example 2 at different time points (3 hours, 6 hours and 15 hours) post application of the test or control articles. The same strain of mice as in Example 2 was used, and the animals were housed in the same manner.

In one set of experimental 8-mouse groups, Gel and Gel+COMPOUND A 3% was applied, then removed 3 hours after application, and the mice subjected to CQ injection at the 3, 6 and 15 hour time points. Control experimental groups with only Gel vehicle applied then removed after 3 hours received either no CQ injection or a CQ injection at the 3 hour time point. To remove the formulation a piece of gauze that had been saturated with distilled water was used to gently wipe (not rub) the application site from the rostral end to the caudal end 3 times. The skin was then patted dry with clean gauze. If there were lesions present on the skin, then extra care is taken to avoid irritation or redness.

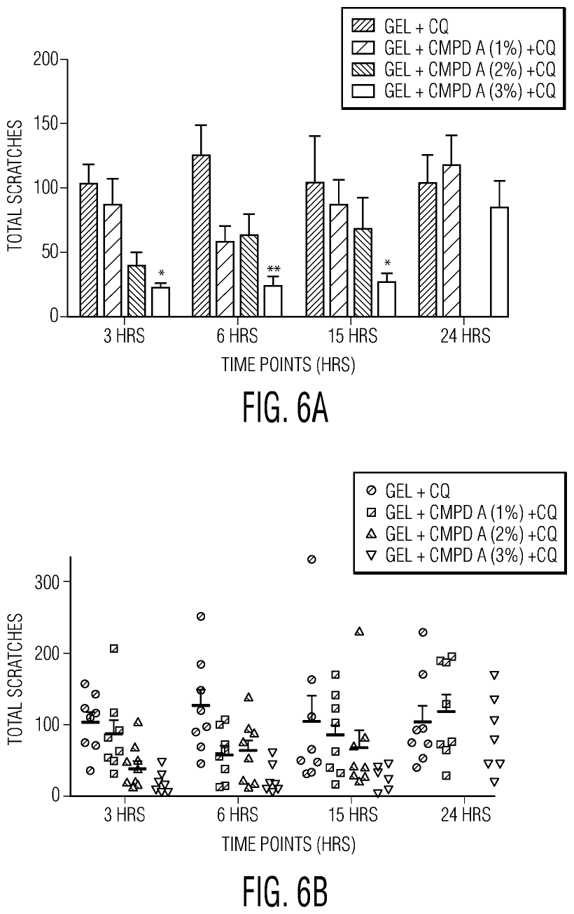

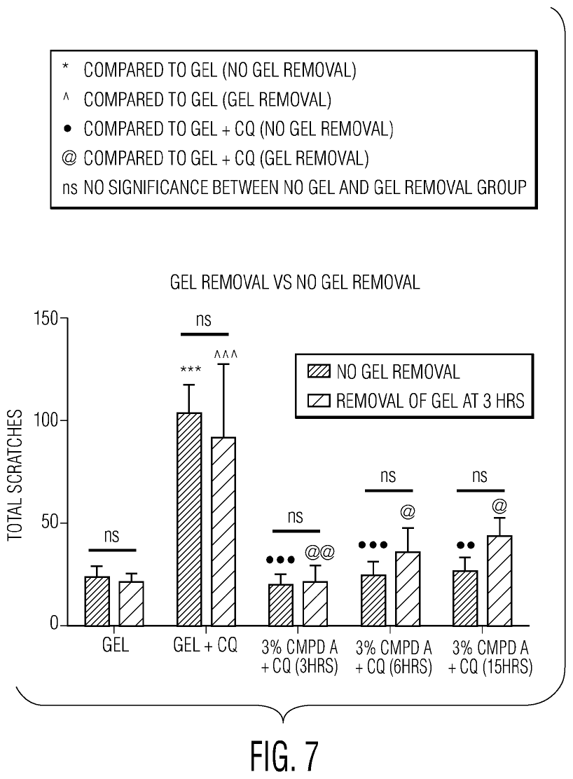

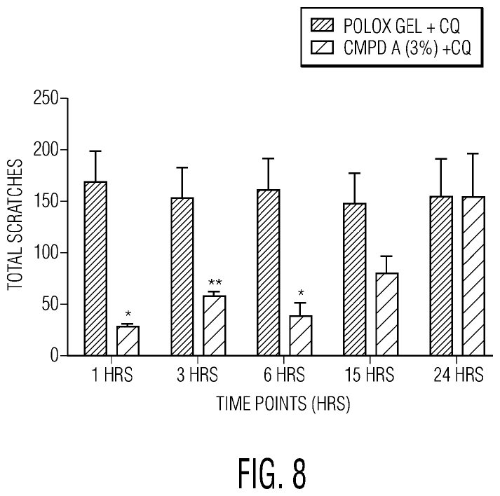

Some mice throughout all groups displayed mild scratching at the site of test article or vehicle application beginning 30 minutes after the application of the test article and lasting for approximately 2 hours. Motor function (measured by placing and stepping) and pinna and corneal reflexes were normal following the application of the drug. No weakness was observed in any of the animals. One mouse in the Gel+COMPOUND A 3% (15 hours) and one from the Gel+COMPOUND A 3% (3 hours) group was removed due to excessive scratching and skin lesions. A few animals in the Gel+COMPOUND A 3% groups showed very mild weakness when handled; however, this weakness did not affect the motor grip strength test and there was no difference in grip strength between the groups in the treated or non-treated groups. Following intradermal CQ injection in the ipsilateral neck area the number of scratches were counted and cumulative counts were recorded by the automated machine for 40 minutes, and the results are shown in FIGS. 6A, 6B and 7.

As can be seen in the figures, application of Gel+COMPOUND A 2% at 3, 6 and 15 hours prior to injection of CQ showed a trend toward a reduction in CQ-induced scratching behavior that was not significantly different from Gel (Vehicle) alone at the same time points. Application of COMPOUND A 3% with removal of test article after 3 hours followed by injection of CQ at 3, 6 and 15 hours showed a significant reduction of scratching behavior when compared to Gel (Vehicle) removed at 3 hours. These results were similar to the reduction in scratching behavior seen with application of COMPOUND A 3% without the removal of the test article. Pre-treatment of animals with Gel+COMPOUND A (2%) did not significantly inhibit chloroquine induced scratches at any of the time points as compared to the group that was pretreated only with the Gel. No differences were observed between Gel+COMPOUND A (1%) and Gel+COMPOUND A (2%) formulations on chloroquine-induced scratching. The animals pre-treated with Gel+COMPOUND A (3%) with or without removal showed similar effects. Animals pretreated with Gel (Vehicle) showed a significant increase in the number of scratches following intradermal chloroquine. The Gel+COMPOUND A (3%) COMPOUND A significantly inhibited chloroquine induced scratching starting at 3 hours and lasted for 15 hours, and this reduction was statistically significant as compared to the group that were pretreated only with the vehicle, irrespective of whether or not the gel was removed 3 hours after application.

### Example 6

### Time Course Study of a Single Application of 3% COMPOUND A Gel in a Mouse Model of Pruritus

To assess the onset time and duration of action of a single application of COMPOUND A topical poloxamer gel, mice were treated with a single application of 3% (2.46% active moiety) COMPOUND A topical gel or placebo, made as described in Example 1, Table 4 above. The mice were then subjected to chloroquine injection (CQ; 4 mg/ml, 50μl) according to the pruritis model described in Example 2 at 1, 3, 6, 15 and 24 hours after application of the gel, and scratches were counted for 40 minutes. The same strain of mice as in Example 2 was used, and the animals were housed in the same manner. Data are summarized in FIG. 7. One hour after application, a robust reduction of chloroquine-induced itch was observed (maximum inhibition observed in this study; p<0.01), indicating an onset time of less than one hour. The reduction in scratching remained robust through 6 hours (p<0.05 at 3 hours; p<0.01 at 6 hours). A partial reduction of chloroquine-induced scratching that did not reach statistical significance in this study was observed at 15 hours, and at 24 hours, scratching behavior of COMPOUND A-treated animals was equivalent to that of the vehicle control animals.

### Example 7

### Double-Blind, Placebo-Controlled, Single Ascending Dose First-In-Human Study

The objective of this study was to evaluate the safety, tolerability, pharmacokinetics and effect on tactile stimulation of topical formulations of COMPOUND A in healthy subjects.

Four single ascending dose cohorts (six active and two placebo subjects per cohort) were evaluated after topical application of a COMPOUND A topical poloxamer gel at an application rate of 82, 164, 328 and 492 μg active moiety/cm² on the volar forearm over a 10 cm² area. Two concentrations of a COMPOUND A topical poloxamer gel: 1% (0.82% active moiety) and 3% (2.46% active moiety) were evaluated in this study. The 1% and 3% gels used were made as described in Example 1 above. Safety was evaluated via hematology, biochemistry and urinalysis clinical laboratory analyses, electrocardiograms and adverse event collection for 14 days following dose administration. The clinical laboratory analyses included hematocrit; Hgb; MCH; MCHC; MCV; MPV; PLT; RBC; WBC and differentials (neutrophils, lymphocytes, monocytes, eosinophils, and basophils relative and absolute); biochemistry albumin; alkaline phosphatase; ALT; AST; chloride; creatinine (enzymatic); GGT; glucose random; LDH; potassium; sodium; total bilirubin; urea (BUN); uric acid; urinalysis color, clarity, pH, specific gravity, bilirubin, glucose, ketones, leukocytes, nitrite, blood, protein, urobilinogen and microscopic analysis. Local skin tolerability was assessed using a 4-point ordinal scale, and serial plasma samples for PK analysis were collected for up to 7 days post-application. The 4-point ordinal scale used was as follows: 0 (None); 1 (Mild); 2 (Moderate); 3 (Severe) for each of the following: erythema, stinging, itching, burning, desquamation, edema, and pain. Semmes-Weinstein microfilaments were used to assess response to tactile stimulation on the treated area of the forearm; these microfilaments were the same as those commonly used to test patients for diabetic neuropathy. If a subject exhibited a tactile response to a certain size of microfilament pre-dose, and after dosing there was no response to that filament and a larger filament was needed for subject to feel the touch, that indicated a loss of some sensation.

A total of 32 subjects (24 active and 8 placebo) were enrolled in this study. All doses of COMPOUND A were well-tolerated. No dose-limiting toxicities or treatment-related adverse events were reported up to the highest administered dose. Excellent local tolerability of the gels was demonstrated with sporadic mild findings. COMPOUND A plasma concentration was below the quantifiable limit of 10 pg/mL in most samples, indicating minimal or no systemic exposure. No trends were noted in tactile stimulation assessments indicating no effect on sensory perception in normal skin. In summary, COMPOUND A topical poloxamer gels exhibited acceptable local and systemic tolerability, minimal systemic exposure and did not affect sensory response to tactile stimulation in normal skin.

While the present disclosure has been discussed in terms of certain embodiments, it should be appreciated that the present disclosure is not so limited. The embodiments are explained herein by way of example, and there are numerous modifications, variations and other embodiments that may be employed that would still be within the scope of the present disclosure.

1. A topical pharmaceutical formulation comprising:
an active ingredient; and
a hydrophilic non-ionic surfactant comprising a poloxamer,
wherein the active ingredient is a compound selected from the group consisting of Formula (I), Formula (II) and Formula (III) or a pharmaceutically acceptable salt or prodrug thereof, 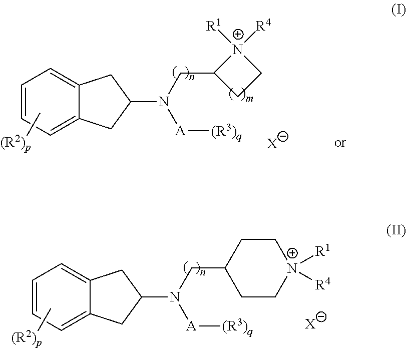
wherein:
A is phenyl or heteroaryl;
R¹ and R⁴ are, independently, C₁ to C₆ alkyl or CH₂CH₂OH; or
R¹ and R⁴ are joined to form a 4- or 6-membered carbocyclic or heterocyclic ring;
R² is independently selected from the group consisting of hydrogen, halogen, NO₂, OH, and C₁ to C₆ alkoxy;
R³ is independently selected from the group consisting of hydrogen, halogen, CN, NO₂, NH₂, optionally substituted C₁ to C₆ alkyl, C₂ to C₆ alkenyl, C₂ to C₆ alkynyl, OH, CF₃, OCF₃, SCF₃, optionally substituted C₁ to C₆ alkoxy, C₂ to C₆ alkynyloxy, heterocyclyloxy, heteroaryloxy, optionally substituted C₁ to C₆ alkylthio, heteroarylthio, C(O)O(C₁ to C₆ alkyl), C(O)(C₁ to C₆ alkyl), C(O)(aryl), C(O)(heterocycle), C(O)NH₂, C(O)NH(C₁ to C₆ alkyl), C(O)NH(aryl), C(O)NH(heterocycle), C(O)NH(heteroaryl), C(O)N(C₁ to C₆ alkyl)(C₁ to C₆ alkyl), C(O)N(aryl)(C₁ to C₆ alkyl), C(S)NH₂, optionally substituted aryl, heteroaryl, heterocycle, NHC(O)(C₁ to C₆ alkyl), NHC(O)(aryl), NHC(O)(heteroaryl), NHC(O)O(C₁ to C₆ alkyl), N(C₁ to C₆ alkyl)C(O)(C₁ to C₆ alkyl), N(C₁ to C₆ alkyl)C(O)O(C₁ to C₆ alkyl), NHC(O)NH₂, NHC(O)NH(C₁ to C₆ alkyl), NHC(O)NH(heteroaryl), NHSO₂(C₁ to C₆ alkyl), SO₂(C₁ to C₆ alkyl), SO₂NH₂, SO₂NH(C₁ to C₆ alkyl), SO₂NH(C₂ to C₆ alkynyl), SO₂N(C₁ to C₆ alkyl)(C₁ to C₆ alkyl), SO₂NH(heteroaryl), NH(C₁ to C₆ alkyl), N(C₁ to C₆ alkyl)(C₁ to C₆ alkyl), N(C₁ to C₆ alkyl)(C₂ to C₆ alkenyl), and N(C₁ to C₆ alkyl)(heterocycle); or
q is 2 and two R³ groups are joined to form an optionally substituted 6-membered aryl, optionally substituted 5- or 6-membered carbocyclic ring, or optionally substituted 5- or 6-membered heterocycle or heteroaryl containing 1 to 3 oxygen, nitrogen, or sulfur atoms and 4 or 5 carbon atoms;
m is 1 to 5;
n is 1 to 3;
p is 0 to 2;
q is 0 to 4; and
X⁻is a halogen ion, trifluoroacetate, sulfate, phosphate, acetate, fumarate, maleate, citrate, pyruvate, succinate, oxalate, bisulfate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, D-mandelate, L-mandelate, propionate, tartarate, phthalate, hydrochlorate, hydrobromate, nitrate, methanesulfonate, ethanesulfonate, napthalenesulfonate, benzenesulfonate, toluenesulfonate, mesitylenesulfonate, camphorsulfonate or trifluoromethanesulfonate, or 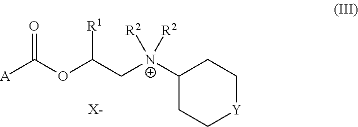
wherein:
R¹ is H or C₁ to C₆ alkyl;
R² is C₁ to C₆ alkyl;
or two R² are joined together to form a 5- or 6-membered ring;
Y is O or CHR³;
R³ is H or C₁ to C₆ alkyl;
A is optionally substituted phenyl, optionally substituted heteroaryl or optionally substituted cycloalkyl, with the proviso that when A is unsubstituted phenyl, R¹ and R² are not methyl and R³ is not H;
X⁻is chloride, bromide, iodide, trifluoroacetate, sulfate, phosphate, acetate, fumarate, maleate, citrate, pyruvate, succinate, oxalate, sulfonate, bisulfate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, D-mandelate, L-mandelate, propionate, tartrate, phthalate, hydrochlorate, hydrobromate, nitrate, methanesulfonate, napthalenesulfonate, benzenesulfonate, toluenesulfonate, camphorsulfonate or trifluoromethanesulfonate.

2. The pharmaceutical formulation of claim 1, wherein the active ingredient comprises about 0.1 to about 10% w/w of the formulation.

3. The pharmaceutical formulation of claim 1, wherein the poloxamer is Kolliphor® P407.

4. The pharmaceutical formulation of claim 1, wherein the poloxamer comprises about 15 to about 40% w/w of the formulation.

5. The pharmaceutical formulation of claim 1, wherein the formulation comprises a topical composition.

6. The pharmaceutical formulation of claim 1, wherein substantially no discoloration and substantially no decrease in viscosity occurs at storage conditions comprising 40° C. and 75% relative humidity.

7. The pharmaceutical formulation of claim 1, wherein the formulation is stable with respect to viscosity at about 40° C. for a period of at least 3 months.

8. The pharmaceutical formulation of claim 1, wherein following storage of the composition for 3 months, under standard storage conditions or accelerated conditions, the total amount of impurities present in the composition is not more than about 3%.

9. The pharmaceutical formulation of claim 1, wherein the active ingredient comprises about 0.1%, 0.3%, 0.5%, 0.7% w/w, 1% w/w or 3% w/w of the formulation.

10. The pharmaceutical formulation of claim 1, wherein the formulation is suitable for transdermal delivery.

11. A method for treating inflammation, pruritis and/or pain, or conditions for which the signs and symptoms include inflammation, pruritis and/or pain, comprising:
topically administering to a subject in need thereof a pharmaceutical formulation, the formulation comprising:
an active ingredient; and
a hydrophilic non-ionic surfactant comprising a poloxamer,
wherein the active ingredient is a compound selected from the group consisting of Formula (I), Formula (II), and Formula (III), or a pharmaceutically acceptable salt or prodrug thereof, 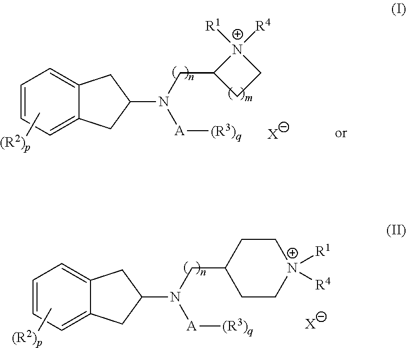
wherein:
A is phenyl or heteroaryl;
R¹ and R⁴ are, independently, C₁ to C₆ alkyl or CH₂CH₂OH; or
R¹ and R⁴ are joined to form a 4- or 6-membered carbocyclic or heterocyclic ring;
R² is independently selected from the group consisting of hydrogen, halogen, NO₂, OH, and C₁ to C₆ alkoxy;
R³ is independently selected from the group consisting of hydrogen, halogen, CN, NO₂, NH₂, optionally substituted C₁ to C₆ alkyl, C₂ to C₆ alkenyl, C₂ to C₆ alkynyl, OH, CF₃, OCF₃, SCF₃, optionally substituted C₁ to C₆ alkoxy, C₂ to C₆ alkynyloxy, heterocyclyloxy, heteroaryloxy, optionally substituted C₁ to C₆ alkylthio, heteroarylthio, C(O)O(C₁ to C₆ alkyl), C(O)(C₁ to C₆ alkyl), C(O)(aryl), C(O)(heterocycle), C(O)NH₂, C(O)NH(C₁ to C₆ alkyl), C(O)NH(aryl), C(O)NH(heterocycle), C(O)NH(heteroaryl), C(O)N(C₁ to C₆ alkyl)(C₁ to C₆ alkyl), C(O)N(aryl)(C₁ to C₆ alkyl), C(S)NH₂, optionally substituted aryl, heteroaryl, heterocycle, NHC(O)(C₁ to C₆ alkyl), NHC(O)(aryl), NHC(O)(heteroaryl), NHC(O)O(C₁ to C₆ alkyl), N(C₁ to C₆ alkyl)C(O)(C₁ to C₆ alkyl), N(C₁ to C₆ alkyl)C(O)O(C₁ to C₆ alkyl), NHC(O)NH₂, NHC(O)NH(C₁ to C₆ alkyl), NHC(O)NH(heteroaryl), NHSO₂(C₁ to C₆ alkyl), SO₂(C₁ to C₆ alkyl), SO₂NH₂, SO₂NH(C₁ to C₆ alkyl), SO₂NH(C₂ to C₆ alkynyl), SO₂N(C₁ to C₆ alkyl)(C₁ to C₆ alkyl), SO₂NH(heteroaryl), NH(C₁ to C₆ alkyl), N(C₁ to C₆ alkyl)(C₁ to C₆ alkyl), N(C₁ to C₆ alkyl)(C₂ to C₆ alkenyl), and N(C₁ to C₆ alkyl)(heterocycle); or
q is 2 and two R³ groups are joined to form an optionally substituted 6-membered aryl, optionally substituted 5- or 6-membered carbocyclic ring, or optionally substituted 5- or 6-membered heterocycle or heteroaryl containing 1 to 3 oxygen, nitrogen, or sulfur atoms and 4 or 5 carbon atoms;
m is 1 to 5;
n is 1 to 3;
p is 0 to 2;
q is 0 to 4; and
X— is a halogen ion, trifluoroacetate, sulfate, phosphate, acetate, fumarate, maleate, citrate, pyruvate, succinate, oxalate, bisulfate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, D-mandelate, L-mandelate, propionate, tartarate, phthalate, hydrochlorate, hydrobromate, nitrate, methanesulfonate, ethanesulfonate, napthalenesulfonate, benzenesulfonate, toluenesulfonate, mesitylenesulfonate, camphorsulfonate or trifluoromethanesulfonate, or 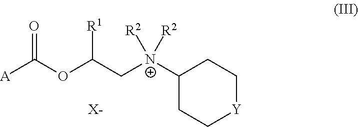
wherein:
R¹ is H or C₁ to C₆ alkyl;
R² is C₁ to C₆ alkyl;
or two R² are joined together to form a 5- or 6-membered ring;
Y is O or CHR³;
R³ is H or C₁ to C₆ alkyl;
A is optionally substituted phenyl, optionally substituted heteroaryl or optionally substituted cycloalkyl, with the proviso that when A is unsubstituted phenyl, R¹ and R² are not methyl and R³ is not H;
X⁻is chloride, bromide, iodide, trifluoroacetate, sulfate, phosphate, acetate, fumarate, maleate, citrate, pyruvate, succinate, oxalate, sulfonate, bisulfate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, D-mandelate, L-mandelate, propionate, tartrate, phthalate, hydrochlorate, hydrobromate, nitrate, methanesulfonate, napthalenesulfonate, benzenesulfonate, toluenesulfonate, camphorsulfonate or trifluoromethanesulfonate.

12. The method of claim 11, wherein the active ingredient comprises about 0.1 to about 10% w/w of the formulation.

13. The method of claim 11, wherein the poloxamer is Kolliphor® P407.

14. The method of claim 11, wherein the poloxamer comprises about 15 to about 40% w/w of the formulation.

15. The method of claim 11, wherein the pharmaceutical formulation comprises a topical composition.

16. The method of claim 11, wherein substantially no discoloration and substantially no decrease in viscosity occurs at storage conditions comprising 40° C. and 75% relative humidity.

17. The method of claim 11, wherein the formulation is stable with respect to viscosity at about 40° C. for a period of at least 3 months.

18. The method of claim 11, wherein following storage of the composition for 3 months, under standard storage conditions or accelerated conditions, the total amount of impurities present in the pharmaceutical formulation is not more than about 3%.

19. The method of claim 11, wherein the active ingredient comprises about 0.1%, 0.3%, 0.5%, 0.7% w/w, 1% w/w or 3% w/w of the pharmaceutical formulation.

20. The method of claim 11, wherein the pharmaceutical formulation is suitable for transdermal delivery.

21. The method of claim 11, wherein an about 80 μg/cm² to about 820 μg/cm²82, 164, 328 or 492 μg/cm² dose of the active ingredient is applied topically to an approximately 10 cm² area of the subject's skin.

22. The method of claim 11, wherein an about 82, 164, 328 or 492 μg/cm² dose of the active ingredient is applied topically to an approximately 10 cm² area of the subject's skin.

23. The method of claim 11, wherein the condition for which the signs and symptoms include inflammation, pruritis and/or pain comprises a dermatological condition.

24. The method of claim 11, wherein the pain comprises chronic pain, neuropathic pain, somatic pain, idiopathic pain, dysfunctional pain, nociceptive pain, neuropathic pain, inflammatory pain, procedural pain, or migraine.

25. The method of claim 11, wherein the condition for which the signs and symptoms include inflammation, pruritis and/or pain is selected from the group consisting of atopic dermatitis, hand and foot eczema, postherpetic itch, dermatitis herpetiformis, postherpetic neuralgia, HIV-associated distal sensory polyneuropathy, prurigo nodularis, pemphigus vulgaris, hypertrophic scar, chronic prurigo, uremic pruritus and notalgia paresthetica.

26. The method of claim 11, wherein hand and foot eczema comprises chronic hand eczema.

27. The method of claim 11, wherein the pharmaceutical formulation is administered twice daily.
